(19) United States
(12) Patent Application Publication
(10) Pub. No.: US 2011/0295083 A1
(43) Pub. Date: Dec. 1, 2011

(54) DEVICES, SYSTEMS, AND METHODS FOR MONITORING, ANALYZING, AND/OR ADJUSTING SLEEP CONDITIONS

(76) Inventors: ERIC N. DOELLING, Sunnyvale, CA (US); Winfried Hohenhorst, Essen (DE); Ryan P. Boucher, San Francisco, CA (US)

(21) Appl. No.: 12/982,475

(22) Filed: Dec. 30, 2010

Related U.S. Application Data
(60) Provisional application No. 61/335,067, filed on Dec. 31, 2009.

(51) Int. Cl. A61B 5/00; A61B 5/05; A61B 5/04; A61B 5/103; A61B 5/053; A61B 5/11; A61B 5/08

(52) U.S. Cl. 600/301; 600/300; 600/595; 600/409; 600/534; 600/587; 600/529; 600/407; 600/373

(57) ABSTRACT

Therapeutic and diagnostic systems and methods help an individual with a sleep disordered breathing condition, such as habitual snoring or obstructive sleep apnea (OSA), achieve deep, restorative sleep. The systems and methods include component that serve complementary sensing, monitoring, and corrective or diagnostic functions.

### RELATED APPLICATION

This application claims the benefit of U.S. Provisional Patent Application Ser. No. 61/335,067, filed Dec. 31, 2009, and entitled “Devices, Systems, and Methods for Monitoring, Analyzing, and/or Adjusting Sleep Conditions.”

### FIELD OF THE INVENTION

The invention generally relates to therapeutic and diagnostics devices, systems, and methods for helping individuals experiencing sleep apnea, snoring, or other forms of sleep obstructive breathing achieve deep, restorative sleep.

### BACKGROUND OF THE INVENTION

Snoring and obstructive sleep apnea (OSA) are common categories of sleep-disordered breathing.

People with untreated OSA stop breathing repeatedly during their sleep, sometimes hundreds of times during the night and often for a minute or longer. Untreated, sleep apnea can cause high blood pressure and other cardiovascular disease, memory problems, weight gain, impotency, and headaches. Moreover, untreated sleep apnea may be responsible for job impairment and motor vehicle crashes. Diagnostic tests for OSA include home oximetry or polysomnography in a sleep clinic.

“Breathing machines” like continuous positive airway pressure (CPAP) may help. The CPAP machine delivers a stream of compressed air via a hose to a nasal pillow, nose mask or full-face mask, splinting the airway (keeping it open under air pressure) so that unobstructed breathing becomes possible, reducing and/or preventing apneas and hypopneas. This has the additional benefit of reducing or eliminating the extremely loud snoring that sometimes accompanies sleep apnea.

Approximately fifty-six percent (56%) of sleep apnea sufferers are position dependent. Position dependent OSA has been defined when an individual experiences at least two times as many apneic events when sleeping in one of the four principal sleeping positions: left side, right side, prone (on the stomach), or supine (on the back).

Snoring, too, is often position dependent and is reduced when a patient changes their position.

### SUMMARY OF THE INVENTION

Some technical features of the invention generally relate to devices, systems, and methods that monitor and/or analyze physiologic and physical conditions of an individual while sleeping.

Other technical features of the invention generally relate to devices, systems, and methods that adjust conditions affecting the physiologic and physical conditions of an individual while sleeping, so that the individual achieves deep, restorative sleep.

Other technical features of the invention generally relate to devices, systems, and methods that make possible the diagnosis and screening in a home setting of individuals who are being denied deep, restorative sleep due to sleep apnea, snoring, or other forms of sleep obstructive breathing.

### BRIEF DESCRIPTION OF THE DRAWINGS

FIG. 1 is a perspective view of a therapeutic system that helps an individual with a sleep disordered breathing condition, such as habitual snoring or obstructive sleep apnea (OSA), achieve deep, restorative sleep, the system including first, second, and third components that serve complementary sensing, monitoring, and corrective functions, respectively.

FIG. 2 is a diagrammatic view of the complementary sensing, monitoring, and corrective functions performed by the system shown in FIG. 1.

FIG. 3A is a perspective view of a gravity-sensitive position sensor that serves a sensing function in the system shown in FIG. 1, being sized and configured to be worn by an individual during sleep.

FIGS. 3B, 3C, and 3D are perspective views of the sensor shown in FIG. 3A in use, being worn on the neck, on a leg, and on a forehead, respectively.

FIGS. 4A and 4B are perspective views of a pressure-sensitive position sensor that serves a sensing function in the system shown in FIG. 1, being sized and configured to be worn by an individual during sleep.

FIG. 4C is perspective view of the sensor shown in FIG. 4A in use, being worn on the neck.

FIG. 5A is a perspective view of a proximity-sensitive position sensor that serves a sensing function in the system shown in FIG. 1, being sized and configured to be worn by an individual during sleep.

FIG. 5B is perspective view of the sensor shown in FIG. 5A in use, being worn on the neck.

FIG. 6A is a perspective view of a magnetic position sensor that serves a sensing function in the system shown in FIG. 1, being sized and configured to be worn by an individual during sleep.

FIG. 6B is perspective view of the sensor shown in FIG. 6A in use, being worn on the neck.

FIGS. 7A, 7B, and 7C are perspective views of an implantable position sensor that serves a sensing function in the system shown in FIG. 1, being sized and configured to be implanted in an individual for use during sleep.

FIGS. 8A and 8B are perspective views of an optical position sensing system that serves a sensing function in the system shown in FIG. 1.

FIGS. 9A and 9B are front and back views, respectively, of a clothing item having an array of different visually distinctive patterns that can be discerned by the optical position sensing system shown in FIGS. 8A and 8B.

FIG. 10 is a diagrammatic view of the complementary sensing, monitoring, and corrective functions performed by the system shown in FIG. 1, including set, pre-programmed rules that establish one or more “best” or desired sleep positions conducive to deep, restorative sleep.

FIG. 11 is a diagrammatic view of the complementary sensing, monitoring, and corrective functions performed by the system shown in FIG. 1, allowing a user or clinician to select among different pre-programmed rules that establish one or more “best” or desired sleep positions conducive to deep, restorative sleep.

FIG. 12 is a perspective and partially diagrammatic view of a component that can serve a monitoring function in the system shown in FIG. 1, which is linked to a remote central station capable of remotely entering the rules that establish one or more “best” or desired sleep positions conducive to deep, restorative sleep.

FIG. 13 is a diagrammatic view of a system like that shown in FIG. 1, which also includes, along with position sensing and monitoring functions, other functions that sense other sleep parameters desired to be monitored.

FIG. 14A is a perspective view of a therapeutic system that helps an individual with a sleep disordered breathing condition, such as habitual snoring or obstructive sleep apnea (OSA), achieve deep, restorative sleep, the system including first, second, and third components that serve complementary sensing, monitoring, and corrective functions, respectively, the sensing function serving to sense respiratory sound during sleep.

FIG. 14B is a diagrammatic view of the complementary sensing, monitoring, and corrective functions performed by the system shown in FIG. 14A.

FIG. 15A is a perspective view of a respiratory sound sensing component that serves a sound-sensing function in the system shown in FIG. 14A, being sized and configured to be worn by an individual during sleep.

FIG. 15B is perspective view of the sensor shown in FIG. 15A in use, being worn about an ear.

FIG. 15C is a perspective view of a respiratory sound sensing component that serves a sound-sensing function in the system shown in FIG. 14A, being sized and configured to be placed beside during sleep.

FIG. 15D is a perspective view of an implantable respiratory sound sensor that serves a sensing function in the system shown in FIG. 14A, being sized and configured to be implanted in an individual for use during sleep.

FIG. 16 is a diagrammatic view of the complementary sensing, monitoring, and corrective functions performed by the system shown in FIG. 14A, including set, pre-programmed rules that establish one or more “best” or desired verbal or nonverbal respiratory sounds conducive to deep, restorative sleep.

FIG. 17 is a perspective and partially diagrammatic view of a system for determining one or more sound architecture benchmarks that can be used in establishing the pre-programmed rules shown in FIG. 16, by correlating endoscopically derived visual images of a breathing obstruction with the auditory architecture of the corresponding verbal or nonverbal respiratory sounds.

FIG. 18 is a diagrammatic view of the complementary sensing, monitoring, and corrective functions performed by the system shown in FIG. 14A, allowing a user or clinician to select among different pre-programmed rules that establish one or more “best” or desired verbal or nonverbal respiratory sounds conducive to deep, restorative sleep.

FIG. 19 is a perspective view of a corrective action element that can perform the corrective function in therapeutic systems like those shown in FIG. 1 or 14A, the element allowing a user to adjust the level of corrective output.

FIG. 20 is a diagrammatic view of the complementary sensing, monitoring, and corrective functions performed by the systems shown in FIG. 1 or FIG. 14A, which further include the capabilities of adjusting the level of or varying the delivery of the corrective output, either locally (as also shown in FIG. 19) or remotely through a remote central station.

FIG. 21 is a perspective view of an active corrective action element that can be controlled by the corrective function of the systems shown in FIG. 1 or FIG. 14A, the corrective action element comprising a controllable sleep surface.

FIG. 22A is a perspective view of an active corrective action element that can be controlled by the corrective function of the systems shown in FIG. 1 or FIG. 14A, the corrective action element comprising a controllable external sleep aid, such as a CPAP machine.

FIG. 22B is a perspective view of an oral device that carries a position and/or sound sensing element.

FIG. 22C is a perspective view of the oral element shown in FIG. 22B that carries a sleep position sensing and correction feedback element.

FIG. 23 is a diagrammatic view of the complementary sensing, monitoring, and corrective functions performed by the systems shown in FIG. 1 or FIG. 14A, which further include the capabilities of selecting among a lists of Apnea Risk Conditions and Corrective Actions and developing for a given individual an optimized response.

FIG. 24 is a diagrammatic view of the Table of Risk Conditions that the functions performed in FIG. 23 rely upon.

FIG. 25 is a diagrammatic view of the Table of Corrective Actions that the functions performed in FIG. 23 rely upon.

FIG. 26 is a perspective view of a diagnostic home screening device for individuals experiencing OSA or other forms of sleep obstructive breathing, which is intended to be a single use or disposable and includes sleep event sensing functions and associated processing functions that monitor and differentiate among conditions of light or no snoring, in which there is no need for clinical concern; heavy snoring, in which making certain sleeping changes may reduce the severity;” and light, moderate or severe OSA, in which consultation with a sleep professional is indicated.

FIG. 27 is a diagrammatic view of a home-based continuous sleep apnea therapy system that continuously guides and monitors an individual experiencing OSA over a prolonged period of time (day-by-day and night-after-night).

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

### I. Therapeutic Devices, Systems, and Methods: an Overview

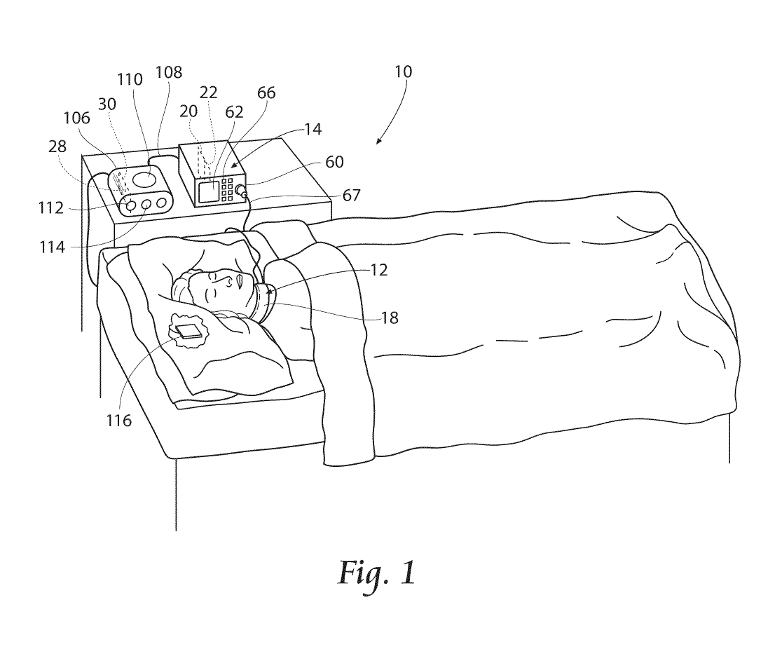

FIG. 1 shows a therapeutic system 10 that helps an individual with a sleep disordered breathing condition, such as habitual snoring or obstructive sleep apnea (OSA), achieve deep, restorative sleep. As shown in FIG. 1, the system includes first, second, and third components 12, 14, 16, respectively. When in data communication, the components 12, 14, and 16 serve complementary sensing, monitoring, and corrective functions, respectively.

A. The First Component (Sensing Function)

The first component 12 is worn by or is otherwise associated with the individual when asleep. In its most basic form, the first component 12 includes a sensing element 18 that senses one or more physical and/or physiologic conditions of the individual while sleeping.

As will be described in greater detail later, the fit, form and function of the first component 12 can vary. The first component 12 can, e.g., be sized and configured to be comfortably worn on the neck, leg, body, head, body extremity, or torso of the individual. Alternatively, the first component 12 can, e.g., be sized and configured to be comfortably integrated into a positive airway pressure mask, e.g., during CPAP. The first component 12 can, alternatively, be sized and configured to be implanted in the individual. Still alternatively, the first component 12 can be sized and configured to be placed at an exterior location, e.g., to visually observe the individual using physical recognition technology, or observe the individual using electronic perception technology (EPT) or radar or sonar-based technologies, or listen to sounds emanating from the individual while asleep.

As will be described in greater detail later, the sensing element 18 can also be variously constructed, depending upon the nature and source of the physical or physiologic condition or conditions that are to be sensed.

The sensing element 18 can, e.g., be position sensitive, to sense a physical sleep position and/or sleep posture of the individual, either with respect to the position of the neck, leg, body, head, body extremity, or torso of the individual, or the position of the head of the individual (rotation, flexion, and/or extension), or both. In this arrangement, the sensing element 18 may sense a gravity position or a pressure condition relative to the sleep surface.

Alternatively, or in combination, the sensing element 18 can, e.g., be sensitive to sound or vibration, to sense breathing sounds the individual makes while sleeping.

Alternatively, or in combination, the sensing element 18 can also be sensitive to physiologic conditions, e.g., peripheral arterial tone; blood pressure; the level of oxygen in the blood (oxygen desaturation/blood saturation); chest and diaphragm effort, expansion and/or contraction; EEG; EMG (electrical muscle activity) heart rate; respiration or breathing rate; arrhythmia detection; periodic cessation of breathing; sleep fragmentation; arousals; sleep state (stage/REM); EOG (measure of REM sleep); measured airflow in and out or vibration of airflow current (e.g., by use of a nasal cannula); nerve signals; airway resistance/flow restriction; positive airway pressure (e.g., CPAP) flow resistance; the pharyngeal critical closing pressure Pcrit; neck tissue compression; and/or

The conditions that are sensed by the sensing element 18 of the first component 12 are desirably pre-established by the manufacturer, a physician or therapist—either based upon individualized data or statistical patient population samples, or both—to reliably differentiate between those physical and/or physiologic conditions that are conducive to or indicative of deep, restorative sleep and those physical and/or physiologic conditions that can interrupt or otherwise interfere with deep, restorative sleep.

The first component 12 can also include the capability for the individual to input information indicative about their own personal condition and sleep conditions. For example, the first component 12 may accommodate the entry of human condition data such as medication being taken, the amount of alcohol consumed, the presence or absence of a sleep partner, and other conditions that may affect the character of the individual's sleep.

Representative embodiments of the first component 12 and its sensing element 18 will be described in greater detail later in Section II.

B. The Second Component (Monitoring Function)

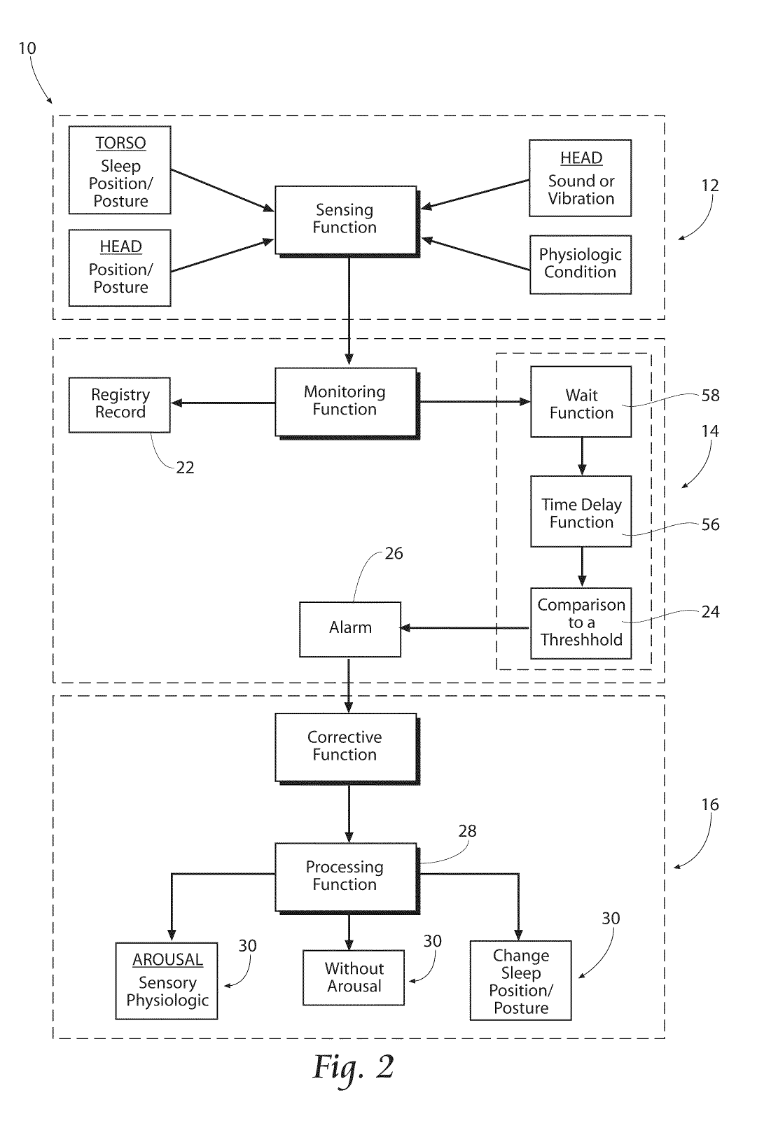

The second component 14 is in data communication with the first component 12. The second component 14 includes a processing element 20 (as diagrammatically shown in FIG. 2), which continuously or periodically registers the condition or conditions sensed by the sensing element 18 or otherwise entered by the individual into the first component 12. The second component 14 can also include a memory element 22 (as also shown in FIG. 2) that retains a record of the sensed conditions, e.g., to create an event log that can be printed, downloaded, or displayed.

As diagrammatically shown in FIG. 2, the processing element 20 desirably includes a comparison function 24, which compares the registered sensed condition or conditions with one or more benchmark or threshold conditions. The benchmark conditions are pre-established—either based upon individual data or statistical patient population samples, or both—to reliable indicate transition between conditions that are conducive to or indicative of deep, restorative sleep and those conditions that signal an actual or potential interruption of sleep.

Physical benchmark conditions can include, e.g., a particular sleep position or posture that is likely to lead to disordered breathing or snoring; and/or the architecture (e.g., amplitude and/or frequency and/or duration) of breathing sounds or snoring that correlates with or is predicative of a disordered breathing or snoring episode. Physiologic benchmark conditions can include, e.g., a specified threshold blood pressure level, or a specified threshold blood oxygen level, or a specified peripheral arterial tone, or a specified heart rate, or a specified breathing rate, or a sudden change in one or more of these physiologic markers, that is undesirable.

The processing element 20 of the second component 14 desirably continuously monitors the sensed conditions in real-time and, by periodic reference to the pre-established benchmarks, automatically signals when the conditions are such that the individual has or is likely to experience an interruption in their sleep cycle. When the sensed condition does not match the benchmark, the processing element 20 desirably generates an alarm output 26 (see FIG. 2). The alarm output 26 is indicative that physical and/or physiologic conditions exist that may interrupt or otherwise interfere with deep, restorative sleep.

The processing element 20 of the second component 14 can also temper the generation of the alarm output 26 by analysis of more than one sensed conditions. In this arrangement, when a first sensed condition does not match its respective benchmark, the alarm output 26 is not generated unless a second sensed condition also does not match its respective benchmark. In this arrangement, the second component 14 receives a sensed first physical and/or physiologic sleep condition of an individual, as well as second sleep condition output indicative of a second physical and/or physiologic sleep condition of the individual different than the first physical and/or physiologic sleep condition. The second component 14 compares the first sleep condition output with one or more benchmark conditions that correlate to a first desired sleep physical and/or physiologic condition, as well as compares the second sleep condition output with one or more benchmark conditions that correlate to a second desired sleep physical and/or physiologic condition. The second component 14 generates an alarm output 26 only when both the first and second desired physical and/or physiologic sleep conditions are absent, thereby tempering the generation of the alarm output 26.

Representative embodiments of the second component 14, its processing element, and its associated functions will be described in greater detail later in Section III.

C. The Third Component (Corrective Function)

The third component 16 is in data communication with the second component 14. The third component 16 includes its own processing element 28 (as diagrammatically shown in FIG. 2), which responds to the alarm output 26 and generates a corrective output. The third component 16 also includes a corrective action function 30 (see FIG. 2) that responds to the alarm output to influence or alter the physical or physiologic conditions of the individual while sleeping, to a return the individual to a sleep state that correlates to the desired benchmark conditions. Return to the benchmark conditions results in the return to deep, restorative sleep. Return to the desired benchmark conditions interrupts the alarm output 26.

As will be described in greater detail later, the corrective action function 30 can be variously constructed or operate in various ways. For example, operation of the corrective action function 30 can affect the individual in a tactile, auditory, or other sensory way sufficient to arouse the individual, thereby teaching or conditioning the individual to alter their sleep position or posture and thereby return to a sleep state more conducive to deep, restorative sleep. Alternatively, and more preferably, operation of the corrective action function 30 can affect the individual on a lower tactile, auditory, or other sensory level that does not awake and/or arouse the individual and/or subconsciously disturb or change or interrupt the sleep state of the individual. In this more preferred arrangement, operation of the corrective action function 30 creates a sensory output having a duration or magnitude that will not necessarily awake and/or arouse the individual and/or subconsciously disturb or change or interrupt the sleep state of the individual, but nevertheless will lead to a subconscious reaction, changing the sleep position or posture or muscle tension in the upper airway. Alternatively, operation of the corrective action function 30 can affect the individual's sleep position or posture by actively altering the orientation or configuration of sleep surface itself. In this arrangement, the corrective action function 30 can articulate or inflate a pillow or a mattress to alter the sleep position or posture of the individual. Alternatively, operation of the corrective action function 30 can control an external sleep aid, such as a therapeutic oral appliance, or positive airway pressure machine (e.g., CPAP), or another device associated with the individual to control physiologic conditions conducive to deep, restorative sleep. Alternatively, the corrective action element 28 can cause a physiologic reaction in the individual, e.g., by tensing a tissue region, or applying pressure to a tissue region, or electrically stimulating a tissue region to prompt the individual to change their sleep position or posture and return to a sleep state more conducive to deep, restorative sleep.

Representative embodiments of the third component 16, its processing element 28, and its associated action function 30 will be described in greater detail later in Section IV. They can include, without limitation, (i) adjustment of positive airway pressure parameters, e.g. CPAP pressure; (ii) injection of smell in positive airway pressure devices; (iii) a time delayed buzzer or vibrator; (iv) the inflation/adjustment of a pillow or mattress; (v) stiffening of tissue; (vi) suspension/application of tension to tissue; (vii) application of pressure to tissue; (viii) adjustment or vibration of oral devices or therapeutic oral appliance such as mandibular advancement devices; (ix) providing sensory stimulation including taste, smell, light, vibration, temperature, nerve or electrical stimulation of muscles; (x) energy stimulation (ultrasound, radio-frequency, etc); (xi) activating a secondary mechanism, i.e. implant, such as a tongue suspension device, a tissue stiffening device; a hyoid suspension device, a genioglosus stimulation device, tissue reshaping device, or other therapeutic devices, or an oral appliance; and (xii) airflow, puffs of air; (xiii) wet sensation. The third component 16 can, if desired, integrate a quick shut-off if the corrective action annoys a sleep partner.

The corrective action function can also generate reports on sleep quality, number and type of corrective actions, out of bound conditions, vital statistics, and effectiveness of sleep interventions for review by the individual and their caregiver. The corrective action function can also correlate and report on the human and environmental conditions affecting the individual as they sleep, such as weight, neck size, pillow type, medications, alcohol consumption, stress, physiologic condition/disposition/state, happiness, sleepiness, pre-sleep activities, room temperature, ambient noise, time of night, sleep stage, and time from onset of sleep. Such reports make it possible for the individual and their caregiver to assess the extent to which these human conditions affect the sleep patterns and sleep quality of the individual.

Illustrative embodiments of therapeutic systems comprising the first, second, and third components 12, 14, and 16 will now be described in the following Sections II, III, and IV, respectively.

### II. Sleep Position Sensing Systems and Methods

Approximately fifty-six percent (56%) of sleep apnea sufferers are position dependent. Position dependent OSA has been defined when an individual experiences at least two times as many apneic events when sleeping in one of the four principal sleeping positions: left side, right side, prone (on the stomach), or supine (on the back). Snoring is often position dependent and is reduced when a patient changes its position.

FIGS. 1 and 2 depict a representative therapeutic system 10 which is sleep position sensitive. The system includes the first, second, and third components 12, 14, and 16, as generally previously described. As diagrammatically shown in FIG. 2, the three components 12, 14, and 16 of the system 10 serve together to perform complementing position sensing functions, position monitoring and alarm functions, and position correction functions, respectively. Functionally, the three components 12, 14, and 16 desirably serve to teach or prompt the individual to assume a “best” or otherwise desired sleeping position, which is defined as the sleep position or positions most conducive to deep, restorative sleep for the individual. As will be described in greater detail later, the “best” or otherwise desired sleep position can be pre-established and fixed, or selectable and adjustable by the user or caregiver, or iteratively established by real-time sleep performance monitoring, or combinations thereof.

A. The Position Sensing Component

As diagrammatically shown in FIG. 2, the first component 12 serves a position sensing function. The position sensed can be a torso or body position, or a head position (rotation, extension, and/or flexion), or both. The position sensing function of the first component 12 can be accomplished in various ways.

1. Gravity-Sensitive Position Sensing

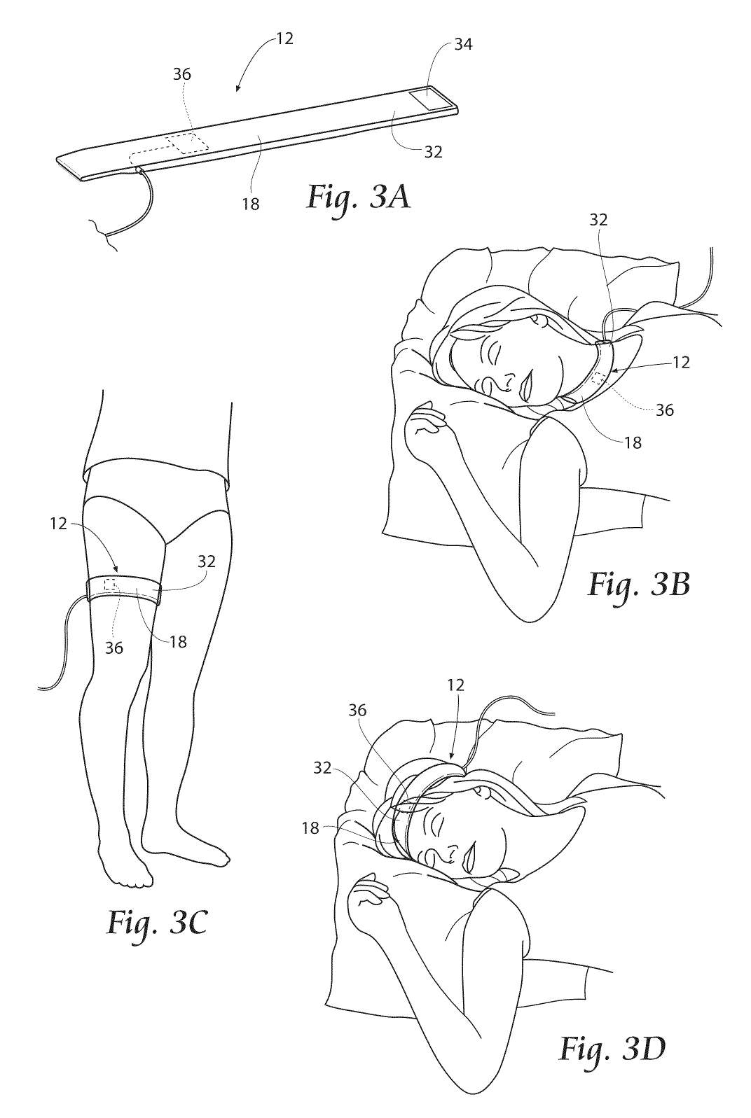

In one embodiment, as shown in FIG. 3A, the first component 12 includes a strap 32 that is sized and configured to be worn about the neck, as FIG. 3B shows. The strap 32 includes suitable fasteners 34 to quickly secure the strap about the neck and release the strap 32 between periods of use. The first component 12 can, alternatively, comprise a sensing element 18 that is carried by a removable adhesive patch, which can be applied prior to sleep and removed after sleep, or which can otherwise be mounted on, integrated to, or affixed to the sleeper.

In the illustrated embodiment, the fasteners comprise VELCRO® Material, but other forms of quick-release fasteners can be used, e.g., snaps, buttons, hooks, etc. Alternatively, the strap 32 can be sized and configured to be worn about a leg or waist (see FIG. 3C), or about the forehead (see FIG. 3D).

In this arrangement (see FIG. 3A), the first component 12 includes, as the sensing element 18, a gravity sensor 36 integrated into the strap 32. The gravity sensor 36 can be of conventional form. For example, it can take the form of a mechanical or electromechanical instrument based on “the bubble in a liquid” concept, like a Mercury gravity switch; or a gravity type potentiometer; or a capacitive gravity sensor; or other forms of miniaturized integrated circuit technologies, such as micro-switches; or forms of micro-electromechanical systems (MEMS).

When the strap 32 is worn about the neck, or elsewhere on the torso (e.g., a leg or about the waist or forehead), the condition of the gravity sensor 36 reflects the relative inclination of the individual's torso. The gravity sensor 36 generates a position dependent output, which is calibrated to change depending upon which sleeping position the individual's torso assumes: left side, right side, prone (on the stomach), or supine (on the back).

When the strap 32 and gravity sensor 36 is worn about the forehead, as shown in FIG. 3D, the position dependent output of the gravity sensor is more particularly indicative of the relative position of the individual's head on the sleeping surface. The position dependent output indicates whether the individual is resting on the left side of the head, right side of the head, back of the head, or face-down.

2. Pressure-Sensitive Position Sensing

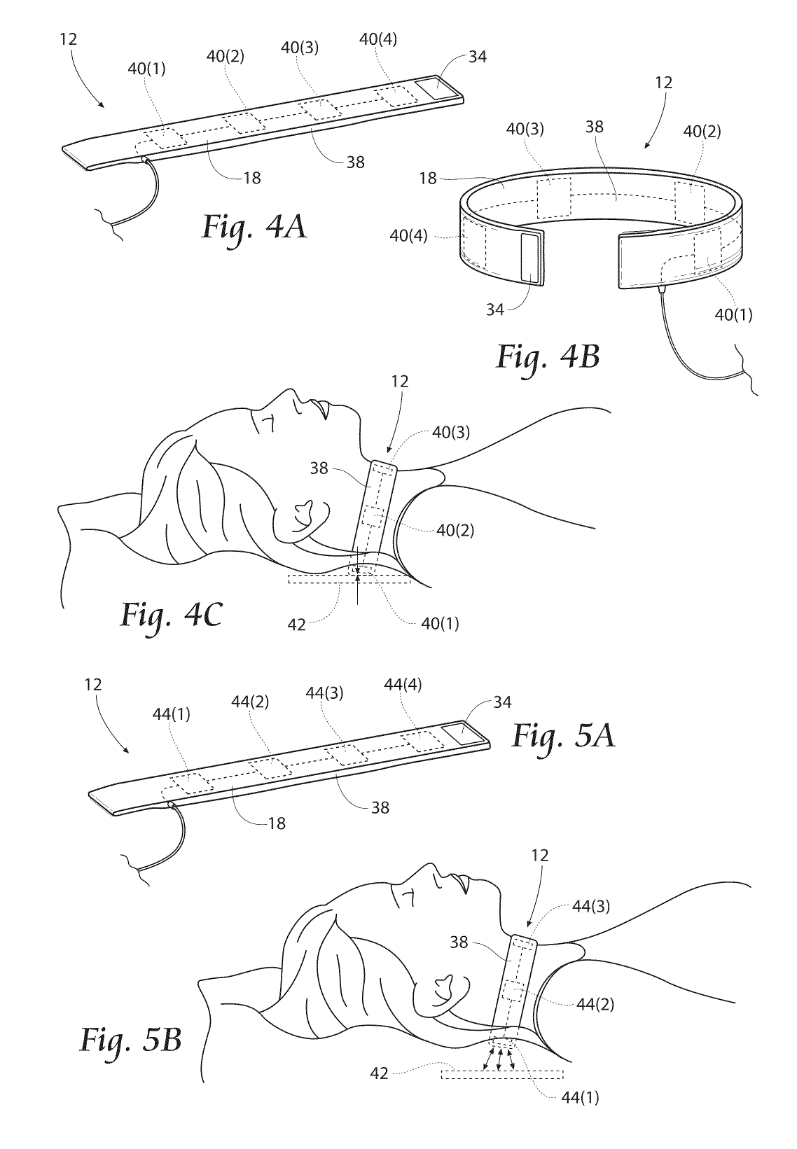

In another embodiment (see FIGS. 4A, 4B, and 4C), the first component 12 can be sized and configured as a carrier 38 to be releasably fitted about the neck, head, or torso, as shown in FIGS. 4A, 4B, and 4C. In this arrangement, the first component 12 carries, as the sensing element 18, an array of pressure-sensitive elements or transducers 40(1), 40(2), 40(3), and 40(4), each of which generates a pressure dependent output when in contact with a sleep surface.

For example, the pressure sensitive elements 40(1) to 40(4) can each comprise pressure sensitive electrical switches that are normally open but close when in contact with a sleep surface 42. Closing the switch generates an electrical pressure dependent output. Pressure sensitive switching can be achieved using electronic devices such as field effect transistors, thyristors, semiconductors, or other forms of miniaturized integrated circuit technologies, such as micro-switches; or forms of micro-electromechanical systems (MEMS).

In this arrangement (see FIGS. 4A and 4B), the array of pressure-sensitive elements 40(1) to 40(4) is distributed about the carrier 38 to correspond with the four primary sleep positions: left side; right side; back; and front. Of course, fewer or more sleep positions may be included. In this arrangement, when the individual is resting on their back on the sleep surface 42 (see FIG. 4C), the corresponding back pressure-sensitive element 40(1) is activated to generate a pressure indicative output, and so on for the other pressure-sensitive elements.

3. Proximity-Sensitive Position Sensing

As another example (see FIGS. 5A and 5B), instead of pressure sensitive elements, the carrier 38 can carry, as the sensing element 18, an array of proximity-sensitive elements 44(1) to 44(4), each of which generates a proximity dependent output when positioned near a sleep surface 42. The proximity-sensitive elements 44(1) to 44(4) detect a sleeping surface 42 by its proximity, without physical contact, as FIG. 5B shows. Various forms of proximity-sensitive elements 44(1) to 44(4) can be used. For example, a capacitive, or photoelectric, or infrared sensor can be used. By placing a metal target in or on the sleeping surface, an inductive proximity sensor can be used. In this arrangement, the array of proximity-sensitive elements 44(1) to 44(4) is distributed about the carrier to correspond with the number of sleep positions which are desired to be monitored: e.g., left side; right side; back; and front. In this arrangement, when the individual is resting on their back, the corresponding back proximity-sensitive element 44(1) is activated to generate a proximity indicative output, and so on for the other proximity-sensitive elements.

4. Magnetic Position Sensing

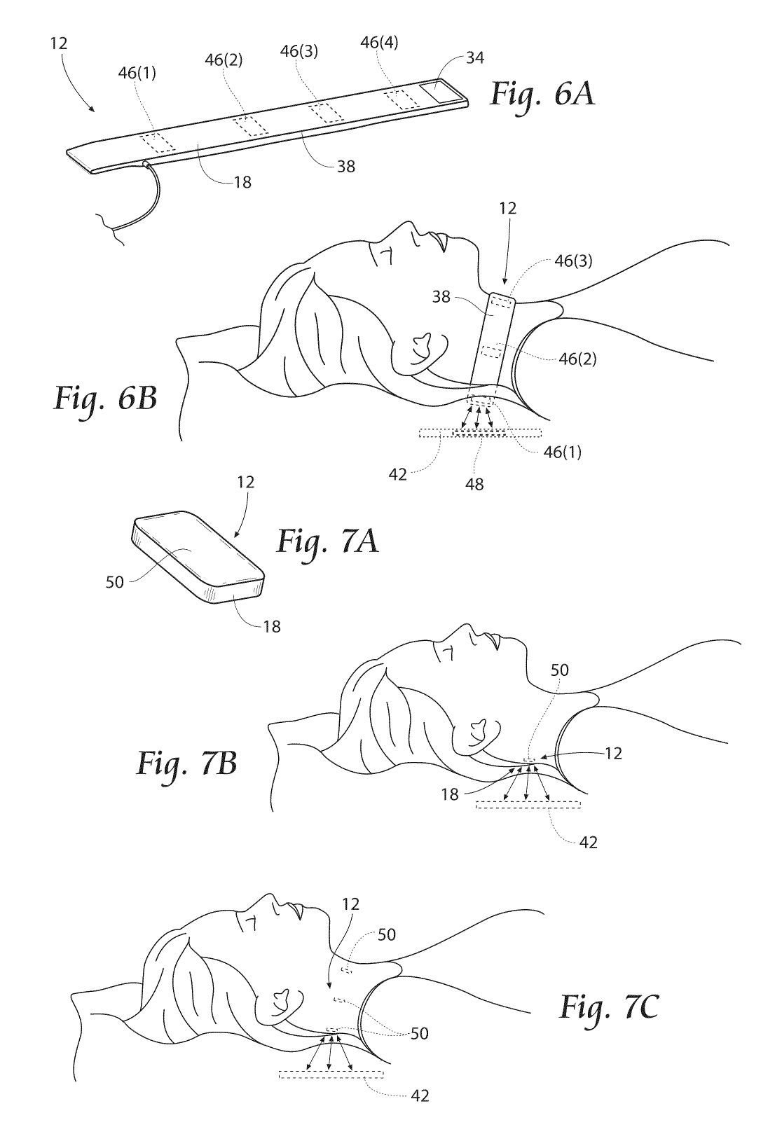

As another example (see FIGS. 6A and 6B), instead of pressure sensitive elements or proximity sensitive elements, the carrier can carry, as the sensing element 18, an array of magnetic sensors 46(1) to 46(4). The magnetic sensors 46(1) to 46(4) can each comprise, e.g., a wire coiled around a permanent magnet. By placing a ferrous metal target 48 in or on the sleeping surface 42, the magnetic sensor will sense the target approaching by sensing changes in magnetic flux through the coil, generating a position indicative voltage at the coil terminals. In this arrangement, as before explained, the array of magnetic sensors 46(1) to 46(4) is distributed about the carrier to correspond with the number of sleep positions which are desired to be monitored: e.g., left side; right side; back; and front. In this arrangement (as FIG. 6B shows), when the individual is resting on their back over the ferrous metal target 48, the corresponding back magnetic sensor generates 46(1) an output, and so on for the other magnetic sensors 46(2), 46(3), and 46(4).

5. Electronic Position-Sensitive Sensors

As another example, the sensing element 18 can employ the position sensing technology of a Wii Remoter™ or Wii MotionPlus™ device, including, e.g., an accelerometer that senses linear motion with or without a dual-axis “tuning fork” angular rate sensor, which can determine rotational motion. This allows for the capture of position and movements that can be analyzed according to pre-programmed rules to ascertain the sleep position of the individual. Based upon this analysis, the processing element 20 generates a position-indicative output.

6. Implantable Position-Sensitive Sensors

In the previous embodiments, the sensing element 18 of the first component 12 is external to the body. In an alternative embodiment, as shown in FIGS. 7A and 7B, the first component 12 comprises one or more sensing elements 50 that are sized and configured for implantation in tissue.

In FIG. 7B, a single implanted sensing element 50 is shown. In this arrangement, the sensing element 50 comprises a gravity sensor incorporated, e.g., in an implantable miniaturized integrated circuit or as a micro-electromechanical system (MEMS).

In another embodiment (see FIG. 7C), an array of sensing elements 50 is implanted in selected tissue regions about the torso to correspond with the number of sleep positions which are desired to be monitored: e.g., left side; right side; back; and front. In this arrangement, the sensing elements 50 can comprise, e.g., a pressure-sensitive element, or a proximity-sensitive element, or a magnetic sensor, as previously described. Whatever the form or function of the implanted sensing element 50, it can take the form of an implantable miniaturized integrated circuit or as a micro-electromechanical system (MEMS).

7. Visual Position Sensing

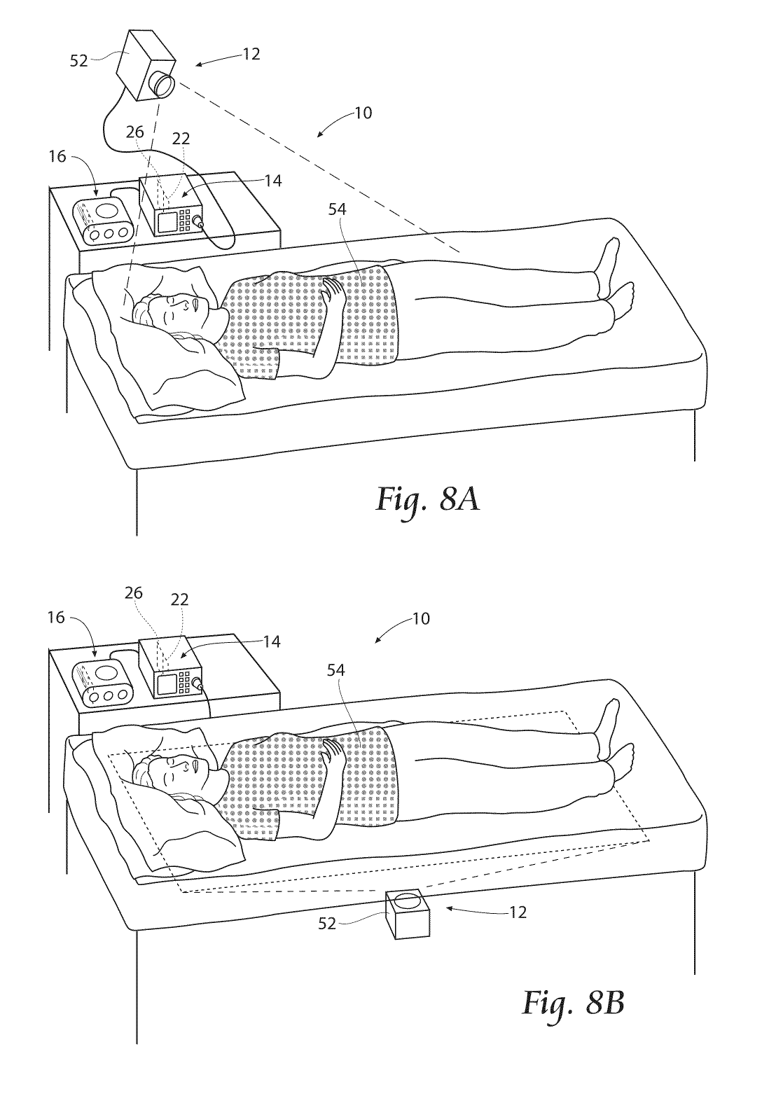

In an alternative embodiment (see FIG. 8A), the first component 12 can comprise an optical monitoring device 52 that captures a visual image of the individual while sleeping. The processing element 20 of the second component 14 is coupled to the monitoring device 52 that analyses the captured visual image according to pre-programmed rules to ascertain the sleep position of the individual. Based upon this analysis, the processing element 20 generates a position-indicative output.

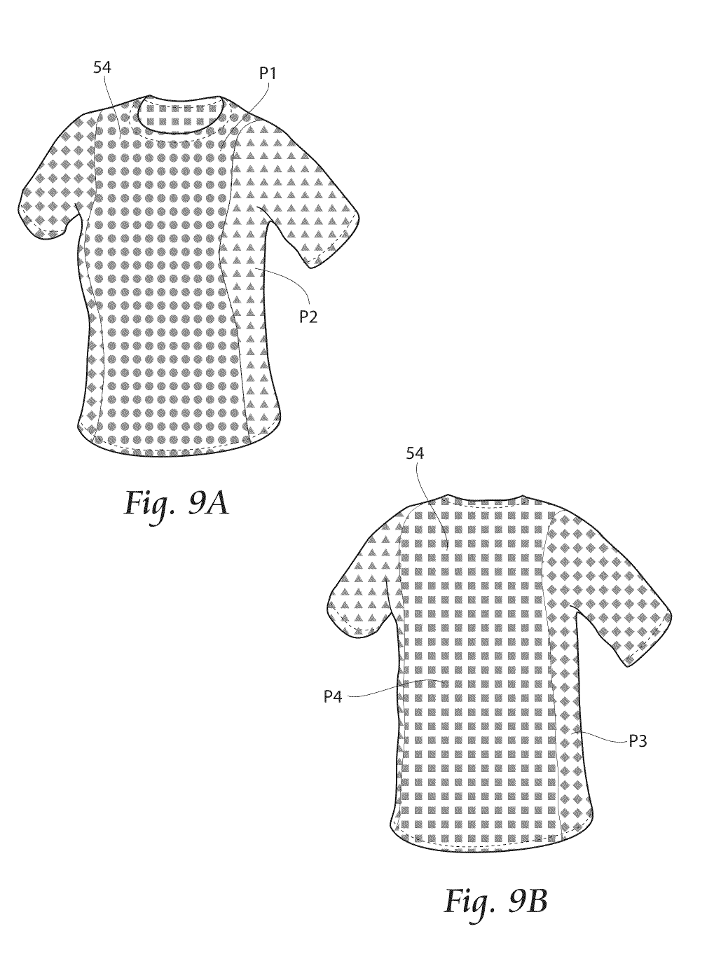

For example, the pre-programmed rules can include a pattern recognition algorithm. In this arrangement (see FIG. 8A), the individual wears on their head or elsewhere on their torso a clothing item 54 that can be viewed by the monitoring device 52. As FIGS. 9A and 9B show, the clothing item 64 includes an array of different visual distinctive patterns P1, P2, P3, P4. The array of patterns p1 to P4 is visually different according to the sleep position of the individual. For example, if the individual is sleeping on their back, an array of circular patterns P4 (see FIG. 9B) is presented to the monitoring device 52. If the individual is sleeping on their left side, an array of square patterns P3 is presented to the monitoring device 52, and so on. The pre-programmed rules of the processing element 20 recognize and differentiate among the different visual patterns and correlates a recognized pattern with a sleep position. The recognized sleep position generates the position-indicative output.

In an alternative embodiment (see FIG. 8B), the sleep surface 42 itself can incorporate a monitor device 52 of the type just described.

Still alternatively, 3-D electronic perception technology (EPT) can be employed to sense the sleep state of an individual, including sleep position. Such technology is available from Microsoft and is described, e.g., in U.S. Pat. Nos. 6,323,942; 6,512,838; and 6,515,740. EPT can be achieved, e.g., using CMOS-based Time-of-Flight [ToF], stereo cameras, and structured light, which is used in mass-market applications such as the XBox™ Kinect™ System. Electronic perception technology enables machines and electronic devices to “see” by tracking nearby objects in three dimensions in real time. Using ToF, e.g., special CMOS chips emit a field of continuous field of infrared light and measure the time it takes for that light to reflect back to the chip—for every pixel. In real-time, the chip processes those distances to create a three dimensional image of the objects in its field of vision. EPT applications can supply actionable information in real time by observing the nearby environment in a reliable, fast, low-cost, and portable form factor, to perceive objects and features in the nearby environment, identify those objects, and take action in real time.

In a representative environment, the first component 12 can comprise an EPT monitoring device that captures a three dimensional image of the individual and their environment while sleeping. The processing element 20 of the second component 14 is coupled to the monitoring device 52 that analyses the captured EPT image according to pre-programmed rules to ascertain the sleep position of the individual. Based upon this analysis, the processing element 20 generates a position-indicative output.

Alternatively, radar or sonar-based technologies can be used to remotely sense and process the sleeping position of an individual and generate a position-indicative output.

The position-indicative output of any position sensor can be correlated with physiological output indicative of the presence or absence of snoring or the presence of absence of obstructed breathing events, or apneas, or hypopneas. The correlation makes it possible to diagnose whether a given individual is position sensitive to such events, and to derive a “best” or otherwise desired sleep position, as will be described later below, and/or to serve as a predictor of an upcoming event so that preventative action can be taken in advance of the event.

8. Oral Device

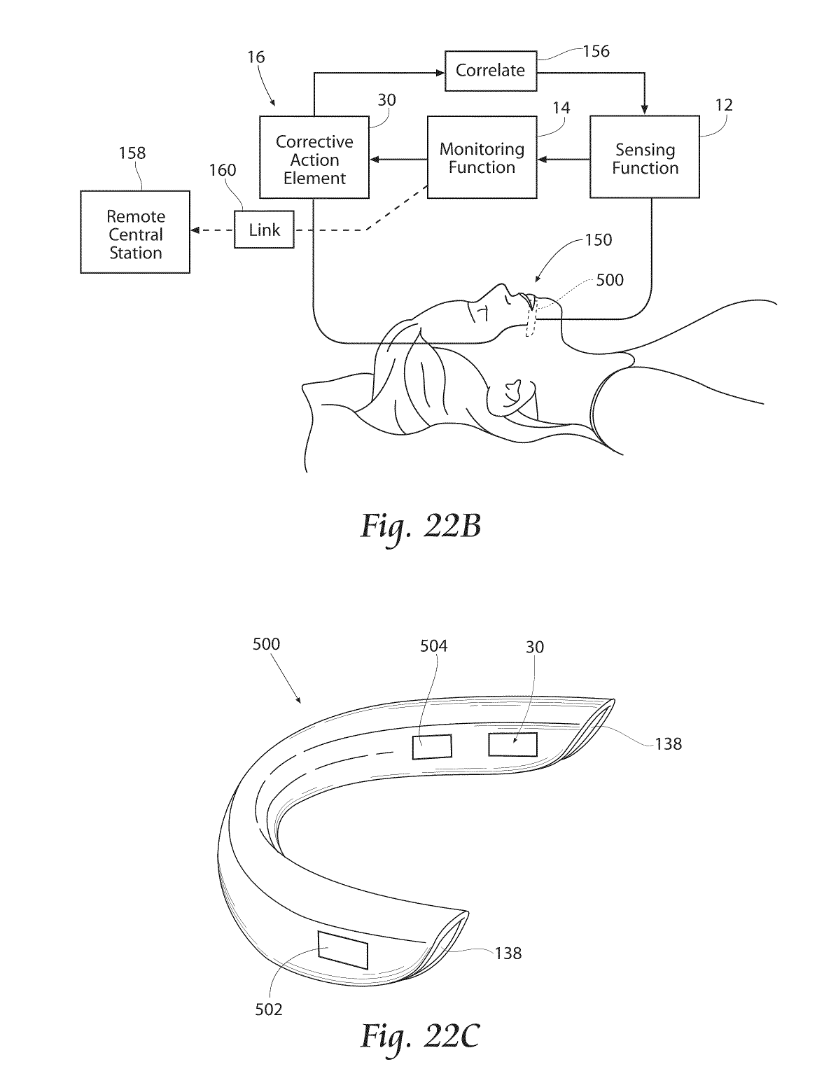

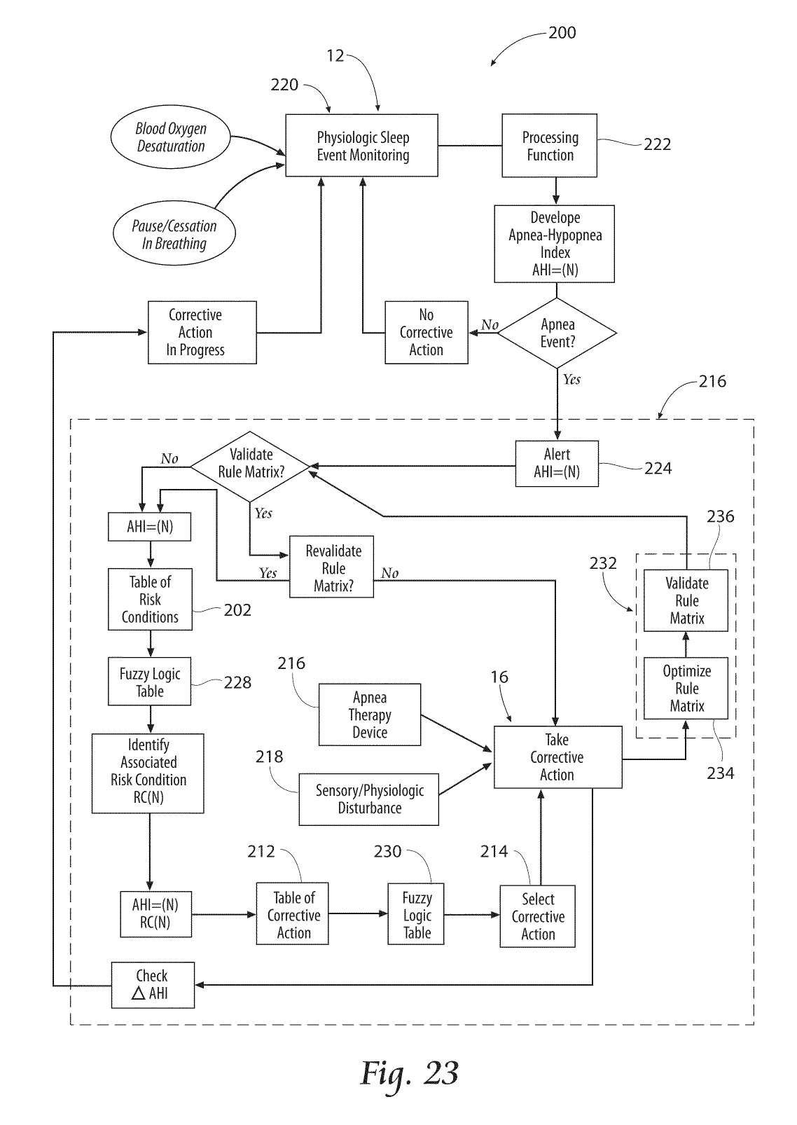

As shown in FIGS. 22B and 22C, a position sensing element 502 is carried by an oral device 500. The oral device 500 comprises a mouth guard, which does not itself perform a therapeutic function, but merely serves to hold the element 12. Desirably, the oral device 500 (see FIG. 22C) is configured for convenient temporary placement into and removal from the oral cavity. Thus, the device 500 may be used only during sleep and removed upon awakening. Removal of the device 500 during waking hours prevents any interference with swallowing, speech, or other routine activities.

The oral device 500 may be constructed in various ways. As shown in FIG. 22C, the device 500 comprises a generally U-shaped body sized and configured to rest in a releasable fit on the lower teeth. The body can also be configured for being worn on the upper teeth. In this arrangement, a sleep position sensing element 502 is carried by the device 500. The position sensing element 502 can take the form of a gravity sensor, e.g., it can take the form of a mechanical or electromechanical instrument based on “the bubble in a liquid” concept, like a Mercury gravity switch; or a gravity type potentiometer; or a capacitive gravity sensor; or other forms of miniaturized integrated circuit technologies, such as micro-switches; or forms of micro-electromechanical systems (MEMS). When the appliance 500 is worn in the oral cavity, the position dependent output of the gravity sensor 502 is indicative of the relative position of the individual's head on the sleeping surface. The position dependent output indicates whether the individual is resting on the left side of the head, right side of the head, back of the head, or face-down.

An overall therapeutic system 10, like that shown in FIGS. 1 and 2, which is sleep-position sensitive can be readily integrated with the device 500. In this arrangement, the sleep position of the individual wearing the device 500 is sensed by the position sensor 502 (i.e., the first component 12) and monitored by the second component 14. The processing element 20 of the second component 14 is pre-programmed to differentiate when the individual's sleep position(s) conform to the “best” or desired sleep position(s) and when they do not. When the individual's sleep position does not conform to the “best” or desired sleep position (s), an alarm output is generated. This functionality will be discussed in greater detail later.

B. The Position Monitoring Component

As diagrammatically shown in FIG. 2, the second component 14 serves a monitoring function. The second component 14 receives the position-indicative output of the first component 12. The second component 14 includes the processing element 20 that continuously or periodically registers the position-indicative output. The processing element 20 includes a comparison function 24, which compares the registered position-indicative output with one or more benchmark or threshold conditions that correlate to a “best” or otherwise desired sleep position. When a correlation is lacking (meaning that the individual is not in the “best” or otherwise desired sleep position), the processing element generates an alarm output.

Desirably (as FIG. 2 diagrammatically shows), the processing element 20 includes a time-delay function 56 that senses the duration of a particular position-indicative output before registering it. In this way, transient changes of position are not registered and processed by comparison function 24. False alarms are thereby eliminated or reduced. The duration of the time delay can be incorporated into the pre-programmed rules and, desirably, be adjusted based upon false alarms experienced.

Desirably (as FIG. 2 diagrammatically shows), the processing element 20 also includes a wait function 58. The wait function 58 delays activation of the comparison function 56 until after a pre-established or pre-set time period within the sleep cycle. The wait function 58 allows enough time for the individual to reach the desired sleep state before the comparison and alarm functions 24 and 26 of the second component 14 are enabled. False alarms are thereby eliminated or reduced at the beginning of the sleep cycle. The duration of the wait function 58 can be incorporated into the pre-programmed rules and, desirably, be adjusted based upon false alarms experienced.

The form and fit of the second component 14 can vary. For example, as seen in FIG. 1, the second component 14 can be incorporated into a compact housing sized and configured to be placed bed-side. The housing 60 desirably houses the processing element 20, which can comprise a microprocessor implemented on an integrated circuit board.

In this arrangement (as FIG. 1 shows), the second component 14 can include a display screen 62. The display 62 can comprise, e.g., a liquid crystal display. The display presents to the individual pertinent operational and status information. In an alternative embodiment, the display screen can be replaced by one or more lighted indicators, e.g., indicating an “on” state; and “off” state; and an “alarm” state.

Communication between the first and second components 12 and 14 can be accomplished by linking the two components with a transmission cable 67 (shown in solid lines in FIG. 1). Alternatively, a wireless communication channel can be established between the two components, e.g., using an infrared transceiver or radio frequency waves including Blue Tooth™ technology. The first component 12 can include on-board memory for storing the position-indicative output, which is downloaded to the second component by direct link or a storage device such as a USB memory device or memory card. The functionality of the second component 14 can be incorporated into a program that can be installed for use on an external computer or personal computer, or as an “app” on a mobile computing device (e.g., an I-Pad™ Device) or cell phone (e.g., an I-Phone™ Device).

The second component 14 can be battery powered, either by use of a standard industry-standard primary battery or an industry-standard rechargeable battery.

The monitoring function of the processing element 20 can be accomplished in various ways.

1. Pre-Set “Best” Sleep Position

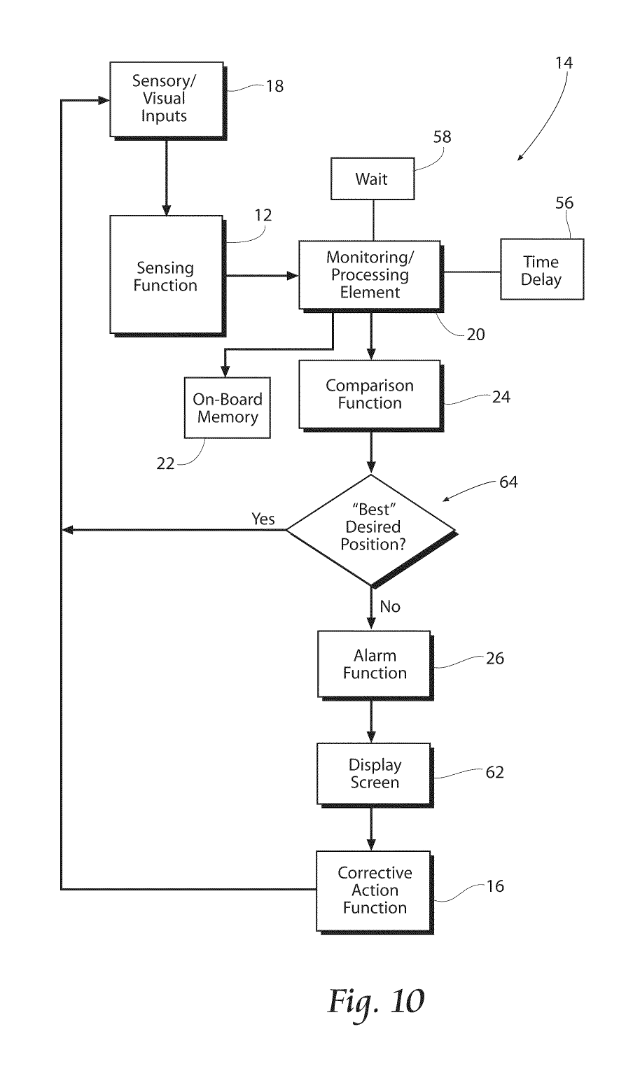

In one embodiment (as diagrammatically shown in FIG. 10), the processing element 20 of the second component 14 can include set, pre-programmed rules 64 that establish one or more sleep positions as the “best” or desired sleep position. The “best” or desired sleep position or positions can be ascertained by diagnosis of individual data (as will be described in greater detail later) or by analysis statistical patient population samples, or both.

For example, a left side and right side sleep position can be pre-programmed in the processing element as being the “best” or desired. These sleep positions thereby become the benchmark conditions.

By continuously or periodically registering the position-indicative output of the first component 12, and by comparing the position-indicative output to the benchmark conditions or rules 64, the processing element of the second component 14 either ascertains a correlation exists (i.e., the individual is resting in a “best” or desired sleep position) or ascertains that a correlation is lacking (i.e., the individual is resting on their back or front). When a correlation is lacking, the processing element generates the alarm output 26. The alarm output 26 can be viewed on the display screen 62. The alarm output 26 is also transmitted to the third component 16 for inducing a change in the sleeping position. Further details of the operation of the third component 16 will be described later in Section IV.

Desirably, as previously described (and as shown in FIG. 1), the processing element 20 carried within the housing 60 includes on-board memory 22 that chronologically stores the position-indicative output overtime for viewing on the display screen 62.

2. Selection of “Best” Sleep Position

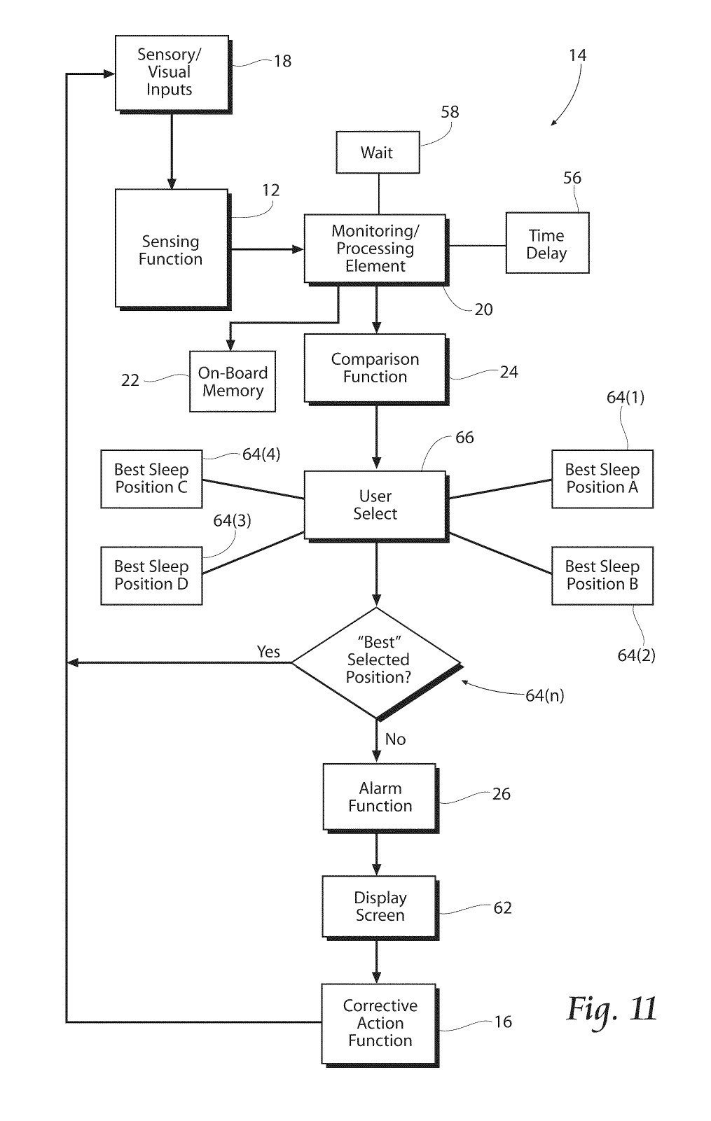

In another embodiment (as diagrammatically shown in FIG. 11), the processing element 20 of the second component 14 can include different pre-programmed rules 64(1), 64(2), 64(3), 64(4) that establish one or more sleep positions as the “best” or desired sleep position. To accommodate this arrangement (see FIG. 1), the second component 14 can comprise both the display screen 62 and a keypad 66, which together form an interactive interface between the individual and the processing element. The display screen 62 presents to the individual pertinent operational and status information, and also prompts the individual to select or modify operational settings using the keypad 66. The keypad 66 can comprise, e.g., a one-piece silicone-rubber molded unit.

In this arrangement, the individual and/or caregiver can, through the keypad 66, select as a “best” or desired sleep position, e.g., a front side position, and/or a left side position, and/or a left side position, and/or a back position, or “anything but a back position” or anything but a front and back position.”

The selected “best” or desired sleep positions thereby become the benchmark conditions of the comparison function 24.

By continuously or periodically registering the position-indicative output of the first component 12, and by comparing the position-indicative output to the selected benchmark conditions, the processing element 20 of the second component 14 either ascertains a correlation exists (i.e., the individual is resting in a “best” or desired sleep position) or ascertains that a correlation is lacking (i.e., the individual is resting on their back or front). When a correlation is lacking, the processing element generates an alarm output 26.

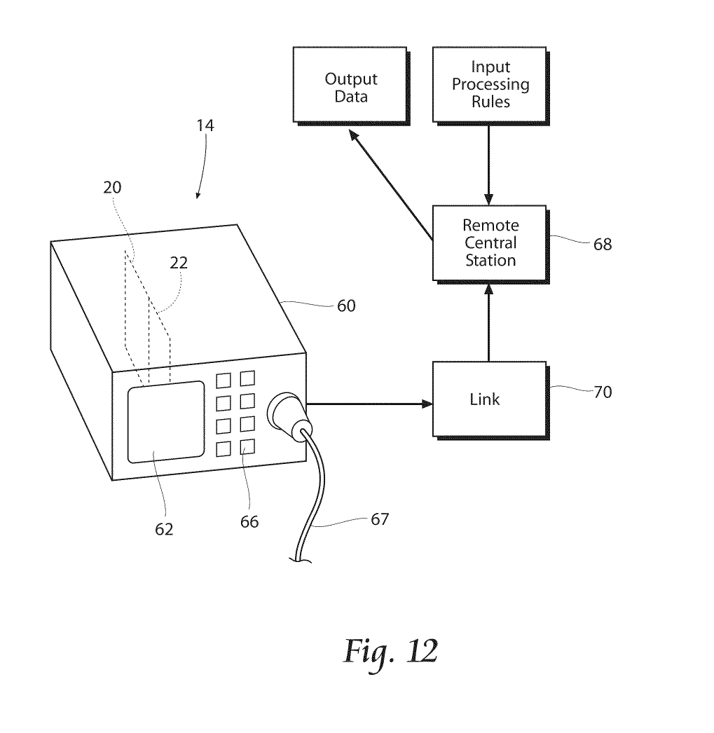

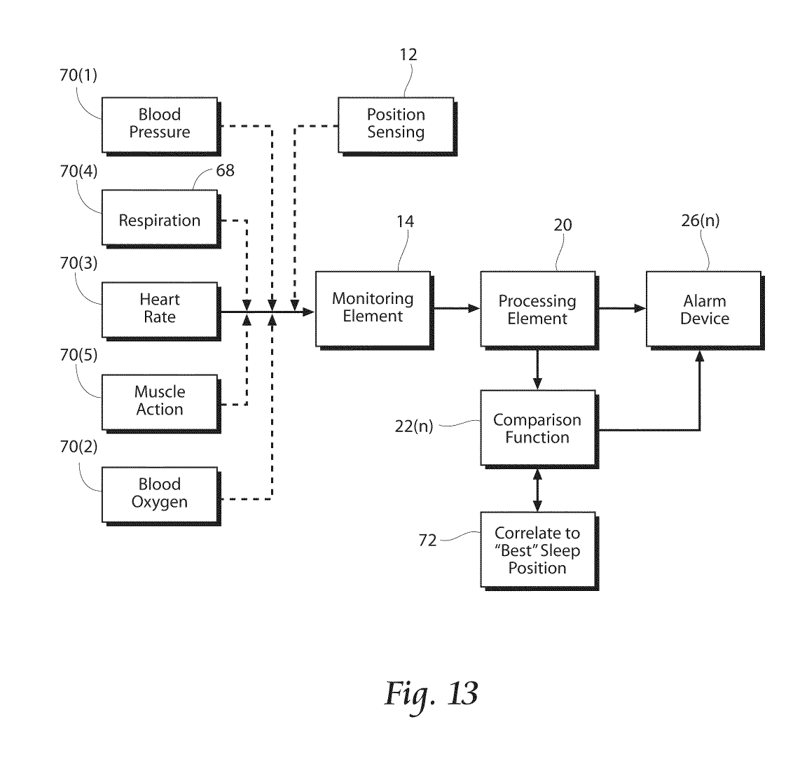

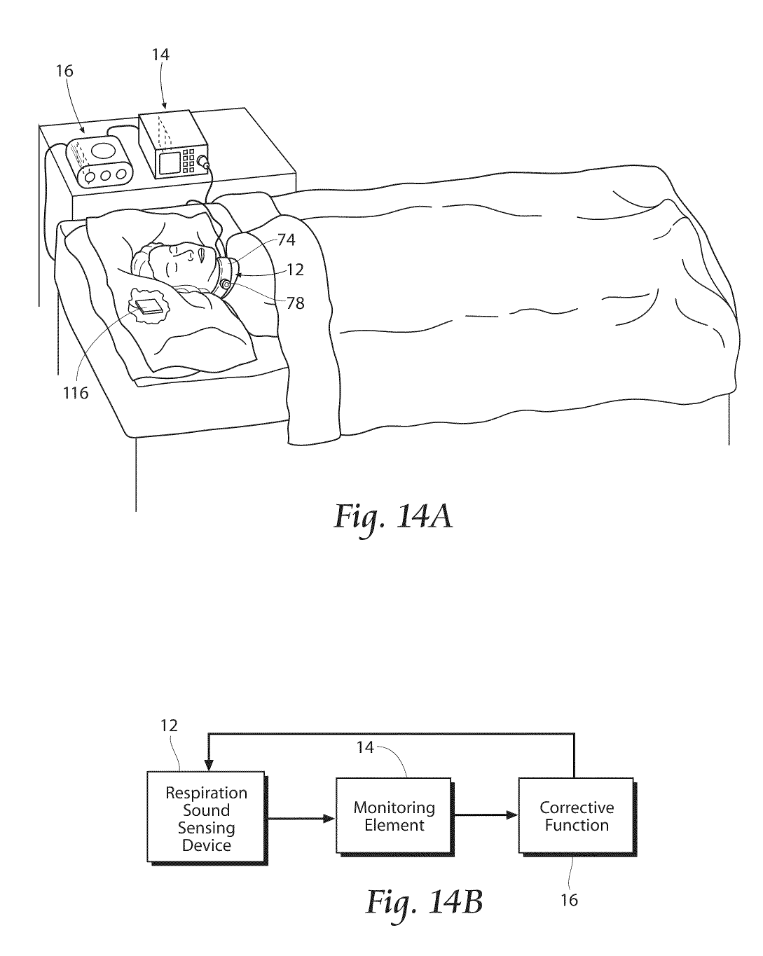

In an alternative embodiment (see FIG. 12), the second component 14 can be linked to a remote central station 68 by landline or internet connection link 70. Through the link 70, caregivers at the central station can review the chronological log the position-indicative output over time, and/or review the selection of “best” sleep positions and, if desired, remotely change them.

3. Iterative “Best” Sleep Position Selection

In another embodiment (see FIG. 13), the system includes, in addition to the position sensing and monitoring functions just described, other components that sense and monitor other sleep parameters of the individual. For example, the other components can include a component 68 that is sensitive to sound or vibration, to sense breathing sounds the individual makes while sleeping. Alternatively, or in combination, the other components can include one or more other body contact or non-body contact components 70(1) to 70(5) that are sensitive to physiologic conditions, e.g., peripheral arterial tone; blood pressure; the level of oxygen in the blood (oxygen desaturation/blood saturation); chest and diaphragm effort, expansion and/or contraction; EEG; EMG (electrical muscle activity) heart rate; respiration or breathing rate; arrhythmia detection; periodic cessation of breathing; sleep fragmentation; arousals; sleep state (stage/REM); EOG (measure of REM sleep); measured airflow in and out or vibration of airflow current (e.g., by use of a nasal cannula); nerve signals; airway resistance/flow restriction; positive airway pressure (e.g., CPAP) flow resistance; the pharyngeal critical closing pressure Pcrit; neck tissue compression; and/or muscle tension/strain.

In this arrangement, the processing element 20 of the second component 14 includes additional comparison functions 22(n) that individually compare the other sensed sleep parameters 12 and 70(1) to 70(5) to pre-established benchmarks and generate individual alarm outputs if the proper correlation does not exist. The processing element 20 therefore generates alarm functions 26(n) from a host of different physical and physiologic sleep parameters, that are not limited to sleep position but to other aspects of the individual's sleep state also conducive to deep, restorative sleep.

In this arrangement (diagrammatically shown in FIG. 13), the processing element 20 can also include a correlation function 72 that, according to pre-programmed rules, correlates the alarm outputs from a host of different physical and physiologic sleep parameters and selects the “best” or desired sleep position based upon the correlation.

For example, assume a sleep session begins with a left side sleep position selected a “best” or desired sleep position. If, during the course of the sleep session, the component sensitive to sound or vibration 68 or 70(4) senses breathing sounds the individual makes while sleeping on their left side that do not correlate to the prescribed benchmark, the correlation function 72 of the processing element 20 can, according to pre-programmed rules, cancel or de-select the left side position from the “best” or desired sleep positions for that sleep session. In this way, the processing element 20 learns and adjusts for the particular events occurring during the individual sleep session. The processing element 20 can continue to learn and adjust in a cumulative fashion during multiple subsequent sleep sessions.

In an alternative embodiment (as previously discussed and as shown in FIG. 12), the second component can be linked to a remote central station 68 by landline or internet connection link 70. Through the link 70, caregivers at the central station 68 can review the chronological log outputs of the various physical and physiologic outputs over time and remotely adjust the selection of “best” sleep positions.

4. Other EPT-Based Systems

As previously described, an EPT sensing component incorporating electronic perception technology (EPT) can be sized and configured to provide a sleep status dependent output indicative of the relative sleep state of the individual. Sleep position, as already described, is a sleep state that can be sensed using an EPT sensing component.

Other conditions affecting an individual sleep state can be sensed using an EPT sensing component. For example, an EPT sensing component can sense the existence of restless leg syndrome; sleep walking; going to and from the bathroom to urinate; tossing, turning, head movement, eye movements due to sleeplessness; GERD-based movements; periodic limb movements; and overall sleep habits. An individual can be provided with a system including an EPT sensing component and instructed to operate the EPT sensing component during a sleep session. The EPT sensing component is sized and configured to observe the sleep state of the individual during the sleep session and generate sleep status dependent output.

A companion monitoring component provided and communicating with the EPT sensing component is sized and configured to compare the sleep status dependent output of the EPT sensing component with one or more benchmark conditions that correlate to a desired sleep state. In this arrangement, the monitoring component generates an alarm output when the individual is not in a desired sleep state.

Further, a corrective action element can be provided to communicate with the monitoring component. The corrective action element generates an output in response to the alarm output. The output influences or alters the individual's sleep state to return the individual to a sleep state that correlates to a desired sleep state. Representative outputs have been previously and will be additionally described, including control of an external controllable sleep aid (e.g., a positive pressure generator or oral appliance); and/or generating at least one sensory or physiologic disturbance; and/or altering an orientation or configuration of a sleep surface.

III. Systems and Methods with Sensing of Sleep Sound Architecture

There is a correlation between the respiratory or breathing sounds an individual makes during sleep and whether or not that individual is in a state of deep, restorative sleep. The architecture of verbal or nonverbal respiratory sounds (e.g., the sound's amplitude, frequency, and duration) changes as the individual transitions from an episode of deep, restorative sleep toward episodes of snoring or episodes of obstructive snoring or episodes of sleep apnea. The architecture of verbal or nonverbal respiratory sounds (e.g., the sound's amplitude, frequency, and duration) can also change depending upon the source of the airway obstruction. Furthermore, different forms of obstructive breathing—e.g., snoring, habitual snoring, sleep apnea, etc.—have different sound architectures.

FIGS. 14A and 14B show a representative therapeutic system 74 which is sensitive to the architecture of an individual's sleeping sounds. The system includes the first, second, and third components, 12, 14, and 16 as generally previously described. In the system shown in FIGS. 14A and 14B, the three components 12, 14, and 16 serve together to perform complementing respiratory sound architecture sensing functions, respiratory sound architecture monitoring and alarm functions, and respiratory sound architecture correction functions, respectively. Functionally, the three components 12, 14, and 16 serve to teach or prompt the individual to sleep in a position or posture where the “best” or desired respiratory sound architecture is achieved, which is defined as the respiratory sound architecture most conducive to deep, restorative sleep for the individual. As will be described in greater detail later, the “best” or otherwise desired sleeping sound architecture can be pre-established and fixed, or selectable and adjustable by the user or caregiver, or iteratively established by real-time sleep performance monitoring, or combinations thereof.

A. The Respiratory Sound Sensing Component

In FIGS. 14A and 14B, the first component 12 serves a respiratory sound sensing function. The respiratory sound sensing function of the first component 12 can be accomplished in various ways.

1. External Respiratory Sound Sensing

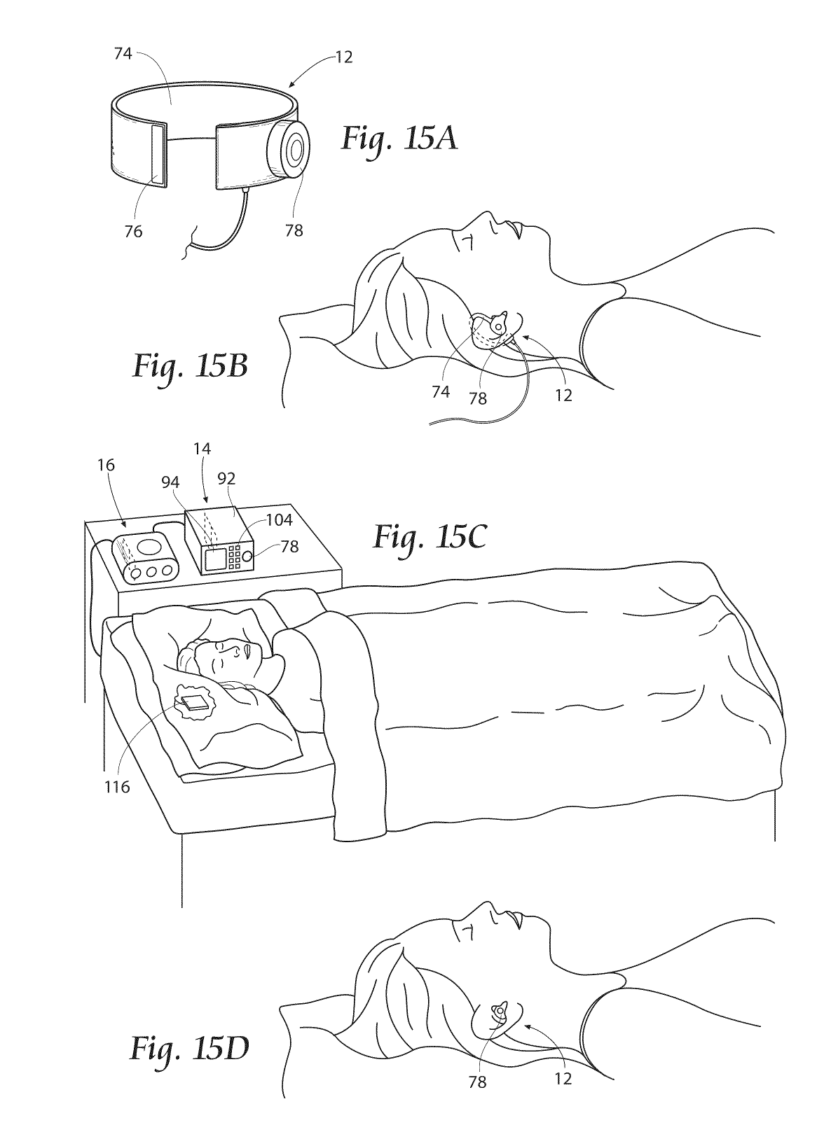

In one embodiment, as shown in FIG. 15A, the first component 12 includes a carrier or strap 74 that is sized and configured to be worn on the body, e.g., about the neck or forehead or on the ear (as FIG. 15B shows). The carrier 74 includes suitable fasteners 76 to quickly secure the carrier about the body and release the carrier between periods of use. In the illustrated embodiment, the fasteners comprise VELCRO® Material, but other forms of quick-release fasteners can be used, e.g., snaps, buttons, hooks, etc.

In this arrangement, the first component 12 includes a sound sensitive element 78 integrated into the carrier 74 for measuring sound energy flow. The sound sensitive element 78 can comprise, e.g., at least one conventional sound sensor, which is also generally referred to as a “microphone.” The first component 12 can, alternatively, comprise a sound sensing element 78 that is carried by a removable adhesive patch, which can be applied prior to sleep and removed after sleep.

Various types of microphones can be used, e.g., dynamic, electrostatic, or piezoelectric. Desirably, the sound sensitive element includes an electrostatic type (condenser) microphone, because it can be downsized, it has generally flat frequency responses over a wide frequency range, and it provides relatively high stability as compared to other types of microphones.

The sound sensitive element can include more than one microphone to measure the sound energy flow. Conventional microphones measure sound pressure (unit: Pa), which represents sound intensity at a specific place (one point), but can measure the direction of flow. A sound intensity microphone is therefore useful for sound source probing and for measuring sound power.

Alternatively, as shown in FIG. 15C, first component 12 can be sized and figured to be placed bed-side or elsewhere in the vicinity of the sleeping individual. Alternatively, the first component 12 can be sized and configured for placement in a pillow or mattress, as will be described later.

The first component 12 generates a sound dependent output which is indicative of the architecture of the verbal or nonverbal respiratory sounds, movements, or vibrations arising during sleep.

2. Implantable Respiratory Sound Sensing

In the previous embodiments, the sound sensitive element is external to the body. In an alternative embodiment, as shown in FIG. 15D, the first component 12 comprises one or more sound sensitive elements 78 that are sized and configured for implantation in tissue.

For example, the sound sensitive element 78 can comprise an implanted microphone sized and configured for placement in an ear, like a hearing aid. The sound sensitive element can comprise an implantable miniaturized integrated sound sensing circuit or a micro-electromechanical sound sensing system (MEMS). In these arrangements, the sound sensitive element can be sized and configured for implantation elsewhere in the body for the detection of verbal and nonverbal respiratory sounds.

3. Oral Device

As shown in FIGS. 22B and 22C, a sound sensitive element 504 can be integrated into an oral device 500 for measuring sound energy flow from within the oral cavity. The oral device 500 has been previously described.

The sound sensitive element 504 can comprise, e.g., at least one conventional sound sensor, which is also generally referred to as a “microphone.” An overall therapeutic system 10, like that shown in FIGS. 1 and 2, which is sleep sound sensitive can be readily integrated with the oral device 500. In this arrangement, the sleep sound architecture of the individual wearing the oral device 500 is sensed by the sound sensitive element 504 (i.e., the first component 12) and monitored by the second component 14. The processing element 20 of the second component 14 compares the monitored sleep sound architecture according to preprogrammed rules or other digital signal processing algorithms with one or more benchmark or threshold conditions that correlate to a “best” or otherwise desired respiratory sound architecture. When a correlation is lacking (meaning that the individual's respiration does not conform to the “best” or otherwise desired architecture), the processing element generates an alarm output. This functionality will be described in greater detail later.

A corrective action element 30 responds to the alarm output. In this arrangement, the corrective action element 30 (coupled to the third component 16) may be also be integrated with the oral device 500. The corrective action element 30 may be variously constructed. As shown in FIG. 22B, the corrective action element 30 comprises, e.g., an electrical buzzer or vibrator 506 carried by the oral appliance 500. The buzzer or vibrator 506 is actuated in response to an alarm output to tactilely or orally disturb the individual. The individual is encouraged and/or taught to change their sleep position and/or posture and/or respiratory pattern to terminate the disturbance, preferably without an abrupt change or disturbance in their sleep state. Alternatively, other forms of sensory disturbance can be activated by the corrective action element 30, e.g., physiologic reaction by the individual, e.g., by applying pressure to the gums fitted to the oral device 500 or electrically stimulating the gums to prompt the individual to change their sleep position or posture or breathing architecture and return to a sleep state more conducive to deep, restorative sleep.

Thus, either a position sensitive component or a sound sensitive component, or both, may be incorporated into an oral device 500. In this way, the system makes possible the maintenance of optimal sleep position and/or posture and/or sleep sound architecture conducive to deep, restorative sleep.

Communication between the sensing and corrective components of the oral device 500 and the external processing elements can be established by interconnecting cables or by wireless signals, such as infrared or radio frequency waves including Blue Tooth™ technology.

In an alternative embodiment (shown in FIG. 22B), the integrated system comprising the oral device 500 and a position and/or sound sensing and monitoring system can be linked to a remote central station 510 by landline or internet connection link 512. Through the link 512, caregivers at the central station 510 can monitor the individual's sleep position and/or posture and/or sleep sound architecture and remotely adjust the magnitude of the corrective action.

B. The Sleep Sound Monitoring Component

Sleep sound architecture means the pattern or signature of the sound energy flow. Sleep sound architecture can be characterized and differentiated in various ways.

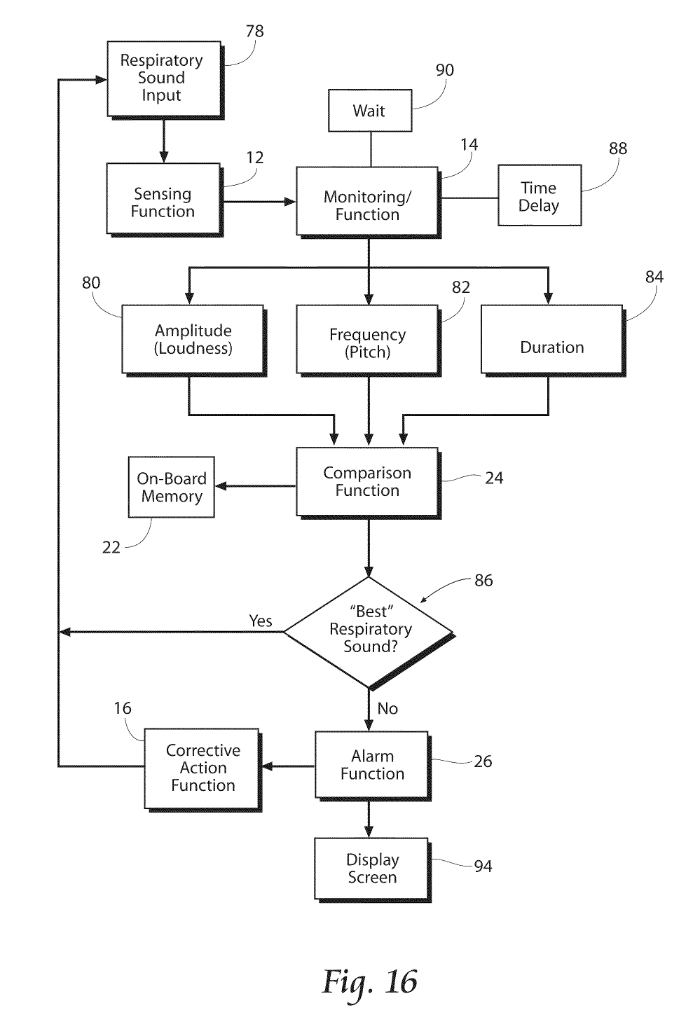

As shown in FIG. 16, one monitored component 80 of sleep sound architecture includes the amplitude or loudness of the verbal or nonverbal respiratory sounds. Snoring or other indications of undesirable or disordered respiration are typically accompanied by sound energy flow characterized with higher peak amplitudes, compared to the peak amplitudes encountered during normal respiration.

As FIG. 16 also shows, another monitored component 82 of the sleep sound architecture includes the frequency or pitch of the verbal or nonverbal respiratory sounds. Snoring or other indications of undesirable or disordered respiration are typically accompanied by sound energy flow characterized with higher or lower frequencies, compared to the frequencies encountered during normal respiration.

As FIG. 16 also shows, another monitored component 84 of the sleep sound architecture includes the duration of the verbal or nonverbal respiratory sounds of a given frequency and/or pitch. The sound architectures of snoring, obstructive snoring, and sleep apnea can be differentiated, at least in part, by the duration of sound signals, as well as by their amplitude and frequency.

In FIG. 16, the second component 14 serves a monitoring function. The second component 14 receives the sound architecture-indicative output of the first component 12. The second component 14 includes the processing element 20 that continuously or periodically registers one or more of the components 80, 82, and 84 comprising the sound architecture-indicative output. The processing element 20 includes a comparison function 24, which compares the monitored components 80, 82, 84 of the registered sound architecture-indicative output according to preprogrammed rules 86 or other digital signal processing algorithms with one or more benchmark or threshold conditions that correlate to a “best” or otherwise desired respiratory sound architecture. When a correlation is lacking (meaning that the individual's respiration does not conform to the “best” or otherwise desired architecture), the processing element generates an alarm output.

The processing element 20 of the second component 14 can also condition the generation of the alarm output by the analysis of more than one sensed conditions. In this arrangement, when a first sensed condition does not match its respective benchmark, the alarm output 26 is not generated unless a second sensed condition also does not match its respective benchmark. For example, the first sensed condition can be position sensitive and the second sensed condition can be sound sensitive, or sensitive to another sensed physiologic condition. In this arrangement, if the position-sensitive condition indicates that an individual is sleeping on their back (or otherwise not in the “best” or desired sleep position), an alarm output is not generated if the sound-sensitive condition (or another physiologic condition) conforms to the “best” or desired sound condition, or vice versa. Thus, the alarm output is generated only if both the position-sensitive condition and the sound-sensitive condition (or another physiologic condition) do not conform to the “best” or desired sound condition.

Desirably, as previously described with respect to sleep position sensing, the processing element 20 includes a time-delay function 88 (see FIG. 16) that senses the duration of a particular sound architecture-indicative output before registering it. In this way, transient changes in sound architecture are not registered and processed by comparison function. False alarms are thereby eliminated or reduced. The duration of the time delay can be incorporated into the pre-programmed rules and, desirably, be adjusted based upon false alarms experienced.

Desirably, as previously described with respect to sleep position sensing, the processing element 20 also includes a wait function 90 (see FIG. 16). The wait function 90 delays activation of the comparison function until after a pre-established or pre-set time period within the sleep cycle. The wait function 90 allows enough time for the individual to reach the desired sleep state before the comparison and alarm functions of the second component 14 are enabled. False alarms are thereby eliminated or reduced at the beginning of the sleep cycle. The duration of the wait function can be incorporated into the pre-programmed rules and, desirably, be adjusted based upon false alarms experienced.

The form and fit of the second component 14 can vary. In form and fit, the second component 14 of the sound-sensitive system 74 shown in FIG. 14A can be like that of the second component 14 of the position-sensitive position shown in FIG. 1, which is shown in FIG. 15C. Thus, the second component 14 can comprise a housing 92 sized and configured to be placed bed-side. The housing 92 houses the processing element 20, which can comprise a microprocessor implemented on an integrated circuit board. The second component 14 of the sound-sensitive system 74 can also include a display screen 94, or the display screen can be replaced by one or more lighted indicators, e.g., simply indicating an “on” state; and “off” state; and an “alarm” state.

As before explained, the monitoring component 14 can communicate with a sound sensitive component 78 worn by a sleeping individual by linking the two components with a transmission cable, in the manner shown in FIG. 1. Alternatively, a wireless communication channel can be established between the two components, e.g., using an infrared transceiver or radio frequency waves including Blue Tooth™ technology. Alternatively, the sound sensitive component can be integrally carried by the bedside housing, as FIG. 15C shows. Alternatively, the sound sensitive component 78 can include on-board memory for storing the sound architecture-indicative output, which is downloaded to the sound monitoring component 14 by direct link or a storage device such as a USB memory device or memory card. The functionality of the sound monitoring component 14 can be incorporated into a program that can be installed for use on an external computer or personal computer, or as an “app” on a mobile computing device (e.g., an I-Pad™ Device) or cell phone (e.g., an I-Phone™ Device).

As before explained, the second component 14 can be battery powered, either by use of a standard industry-standard primary batter or an industry-standard rechargeable battery.

The monitoring function of the sound-sensitive processing element can be accomplished in various ways.

1. Determining the “Best” Respiratory Sound Architecture

The “best” or desired respiratory sound architectures can be ascertained by diagnosis of individual data (as will be described in greater detail later) or by analysis statistical patient population samples, or both.

For example, the “best” or desired sleep sound architecture can be ascertained for the individual by screening in a conventional clinical setting. Alternatively, statistical analyses patient population samples can be used to ascertain a “best” or desired sleep sound architecture.

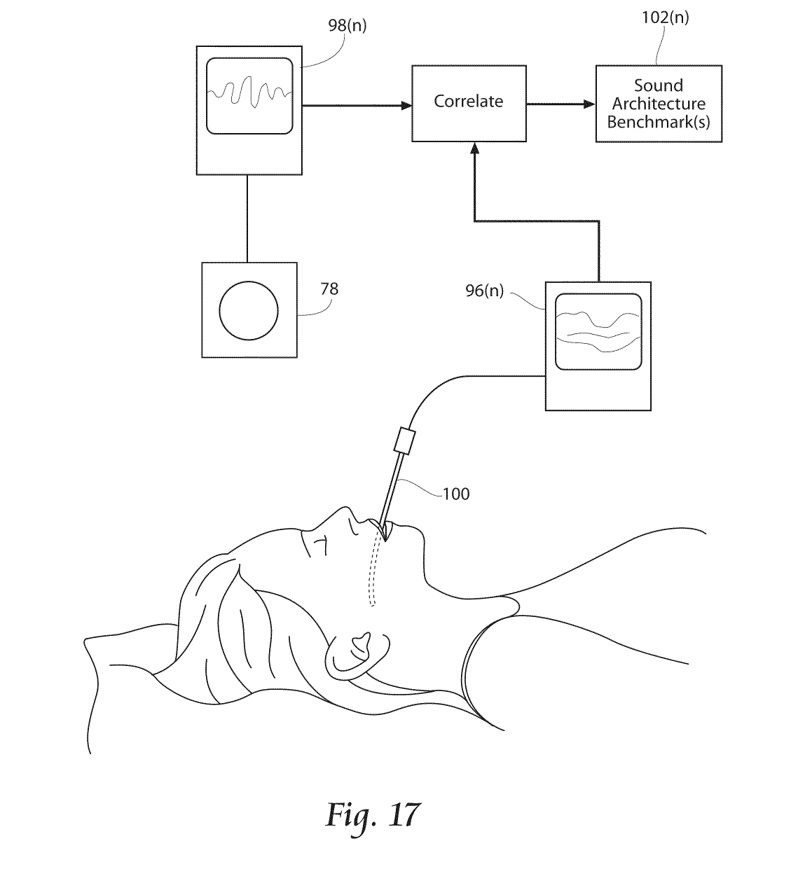

Alternatively, the “best” or desired sleep sound architecture can be obtained by a system and method different than conventional clinical screening, as shown in FIG. 17.

The system and method comprise conducting sleep endoscopy (using an endoscopic optical device 100) on an individual to capture a visual image 96 identifying a site of breathing obstruction. The system and method measure, concurrent with the creation of the visual image 96, the particular respiration sound energy flow 98, monitored by a sound sensitive element 78, to ascertain the sound architecture associated with the visual image.

The system and method correlate the visual image 96, which is particular to the site of breathing obstruction, with the particular sound architecture 98. The system and method derive from the correlation a sound architecture benchmark 102. The system and method desirably derive a plurality of sound architecture benchmarks 102(n) by correlating a plurality of different visual images 96(n) obtained through sleep endoscopy with the particular sound architectures 98(n) taken concurrent with the endoscopy. The plurality of sound architecture benchmarks 102(n) provide the ability to differentiate between different types of disordered breathing sounds and, from these, derive one or more “best” or desired sleep sound architectures.

The method performed by the system can be conducted on a single individual to determine a customized “best” or desired sleep sound architecture for the individual. Statistical analyses of the method conducted on different patient population samples can also be used to ascertain a “best” or desired respiratory sound architecture.

2. Pre-Set “Best” Respiratory Sound Architecture

In one embodiment (as shown in FIG. 16), the sound-sensitive processing element 20 of the second component can include set, pre-programmed rules 86 that establish one or more respiratory sound architectures as the “best” or desired respiratory sound architectures. As just described, the “best” or desired respiratory sound architectures can be ascertained by diagnosis of individual data or by analysis statistical patient population samples, or both.

By continuously or periodically registering the sound architecture-indicative output of the first component 12, and by comparing the sound architecture-indicative output to the benchmark conditions according to pre-programmed rules 86, the processing element 20 of the second component 14 either ascertains a correlation exists (i.e., the individual's respiratory conforms to a “best” or desired respiratory sound architecture) or ascertains that a correlation is lacking (i.e., the individual's verbal or nonverbal respiratory sounds do not conform to the “best” or desired sound architecture). When a correlation is lacking, the processing element 20 generates an alarm output 26. The alarm output 26 can be viewed on the display screen 94 (as shown in FIG. 15C). The alarm output 94 is also transmitted to the third component 16 for inducing a change in the respiratory sound architecture. Further details of the operation of the third component 16 will be described later in Section IV.

Desirably, the processing element 20 includes on-board memory 22 (shown in FIG. 15C) that chronologically stores the sound architecture-indicative output overtime for viewing on the display screen.

3. Selection of “Best” Sleep Sound Architecture

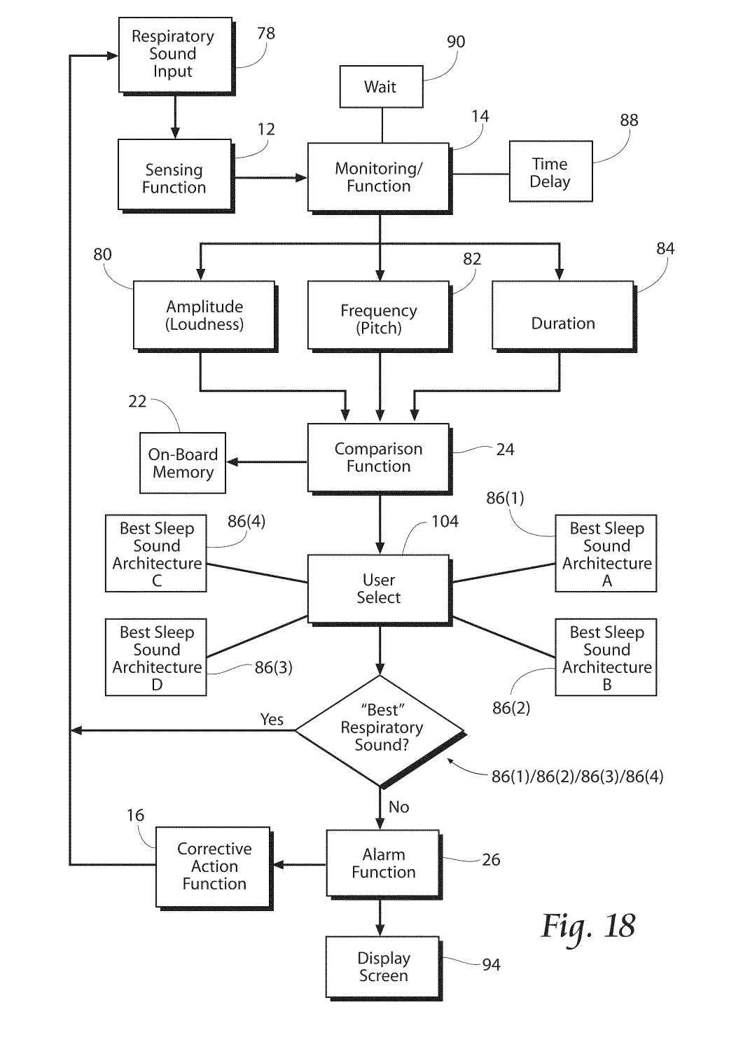

In another embodiment (see FIG. 18), the processing element of the second component 14 can include different pre-programmed rules 86(1) to 86(4) that establish one or more sleep sound architectures as the “best” or desired sleep sound architecture. In this arrangement, the second component 14 comprises both the display screen 94 and a keypad 104 (shown in FIG. 15C), which together form an interactive interface between the individual and the processing element 20. The display presents 94 to the individual pertinent operational and status information, and also prompts the individual to select or modify operational settings using the keypad 104. The keypad 104 can comprise, e.g., a one-piece silicone-rubber molded unit.

In this arrangement, the individual and/or caregiver can, through the keypad 104, select one or more “best” or desired sound architectures 86(1) to 86(4), or “anything but a particular sound architecture.”

The selected “best” sound architecture(s) thereby become the benchmark conditions.

By continuously or periodically registering the sound architecture-indicative output of the first component 12, and by comparing the architecture-indicative output to the selected benchmark conditions 86(1) to 86(4), the processing element 20 of the second component 14 either ascertains a correlation exists (i.e., the individual's respiration sounds conforms to the “best” or desired sound architecture) or ascertains that a correlation is lacking (i.e., the individual's respiration sounds do not conform to the best” or desired sound architecture). When a correlation is lacking, the processing element generates an alarm output.

In an alternative embodiment (as previously described and as shown in FIG. 12), the second component 14 can be linked to a remote central station 68 by landline or internet connection link 70. Through the link 70, caregivers at the central station 68 can review the chronological log the sound architecture-indicative output over time, and/or review the selection of “best” sound architecture and, if desired, remotely change them.

4. Iterative “Best” Sleep Sound Architecture

Referring back to FIG. 13, the system there illustrated includes, in combination with the position sensing and monitoring functions previously described, the sound architecture sensing and monitoring functions just described. The system can also includes other body contact or non-body contact components that sense and monitor other sleep parameters of the individual, e.g., peripheral arterial tone; blood pressure; the level of oxygen in the blood (oxygen desaturation/blood saturation); chest and diaphragm effort, expansion and/or contraction; EEG; EMG (electrical muscle activity) heart rate; respiration or breathing rate; arrhythmia detection; periodic cessation of breathing; sleep fragmentation; arousals; sleep state (stage/REM); EOG (measure of REM sleep); measured airflow in and out or vibration of airflow current (e.g., by use of a nasal cannula); nerve signals; airway resistance/flow restriction; positive airway pressure (e.g., CPAP) flow resistance; the pharyngeal critical closing pressure Pcrit; neck tissue compression; and/or muscle tension/strain.

As previously described, in this arrangement, the processing element 20 of the second component 14 includes additional comparison functions 22(n) that individually compare the other sensed sleep parameters 12, 68, and 70(1) to 70(5) to pre-established benchmarks and generate individual alarm outputs 26(n) if the proper correlation does not exist. The processing element therefore generates alarm functions from a host of different physical and physiologic sleep parameters, that are not limited to sleep sound architecture but to other aspects of the individual's sleep state also conducive to deep, restorative sleep.

In this arrangement, the processing element also includes a correlation function 72 that, according to pre-programmed rules, correlates the alarm outputs from a host of different physical and physiologic sleep parameters and selects the “best” or desired sleep position based upon the correlation. For example, assume a sleep session begins with prescribed sleep sound architecture selected a “best” or desired sleep sound architecture. If, during the course of the sleep session, the component sensitive to sleep position does not correlate to the prescribed benchmark, the correlation function of the processing element can, according to pre-programmed rules, cancel or de-select the prescribed sound architecture from the “best” or desired sleep sound architecture for that sleep session. In this way, the processing element learns and adjusts for the particular events occurring during the individual sleep session. The processing element can continue to learn and adjust in a cumulative fashion during multiple subsequent sleep sessions.

In an alternative embodiment (see FIG. 12), the second component 14 can be linked to a remote central station 68 by landline or internet connection link 70 or suitable telemedicine link (the term “telemedicine” broadly defined to mean “the transfer of medical information via telecommunication technologies for the purpose of consulting or for remote medical procedures or examinations”) (which in shorthand will be call “the “tele-med link”). Through the link 70, caregivers at the central station 68 can review the chronological log outputs of the various physical and physiologic outputs over time and remotely adjust the selection of “best” sleep sound architecture accordingly.

Alternatively, as previously described, the “best” or desired sleep position can be iteratively adjusted based upon correlation by pre-programmed rules with sleep sound architecture-indicative output. Or alternatively, the correlation function can incorporate “best fit” rules to optimize both sleep sound architecture and sleep position.

### IV. The Corrective Component

In each of the systems already described, the third component 16 is coupled to the second component. The third component 16 includes a processing element 28 that responds to the respective alarm output (based either upon sleep position, or sleep sound architecture, or another measured sleep parameter, vital sign, or physiologic parameters, or combinations thereof, and generates a corrective output.

The third component 16 also includes a corrective action element 30 that responds to the alarm output to influence or alter the individual's sleep position, or sleep sound architecture, or both, to a return the individual to a sleep state that correlates to the desired benchmark conditions. Return to the benchmark conditions results in the return to deep, restorative sleep Return to the desired benchmark conditions interrupts the alarm input.

As shown in FIG. 1, in any one of the systems described, the third component 16 can comprise a housing 106 sized and configured to be placed bed-side separate from the second component 14. The housing 106 desirably houses the processing element 28 of the third component 16, which can comprise a microprocessor implemented on an integrated circuit board. Communication between the second and third components 14 and 16 can be accomplished by linking the two components with a transmission cable 108 (shown in FIG. 1). Alternatively, a wireless communication channel can be established between the two components, e.g., using an infrared transceiver or radio frequency waves including Blue Tooth™ technology. Alternatively, the second and third component 16 can be incorporated or fully integrated incorporated into a single housing.

The corrective action element 30 of the third component 16 can vary in construction and function.

A. Sensory Corrective Action

As shown in FIG. 1, the corrective action element 30 can be coupled to a speaker 110 through which an audible sound is generated to awake the individual. The individual, now aroused, is forced to change his sleeping position to silence the sound.

As also shown in FIG. 1, the corrective action element 30 can also include a panel 112 through which visual light is pulsed to awake the individual, alone or in concert with the audible sound through the speaker 110. The individual, now aroused, is forced to change his sleeping position to silence the sound and/or light and return to sleep. Alternative, or in combination with one or more of the other sensory outputs, the corrective action element can include an opening 114 through which a gaseous odor is sprayed to awake the individual.

As shown in FIG. 1, the corrective action element 30 can comprise an electrical buzzer or vibrator 116 placed on the sleep surface, attached to the clothing of the individual, or integrated in the carrier for the sensing device. The corrective action element 30 actuates the buzzer or vibrator to tactilely disturb the individual to awake him/her. The individual, now aroused, is forced to change his sleeping position to terminate the disturbance and return to sleep.

Other forms of sensory disturbance can be activated by the corrective action element 30, e.g., by a physiologic reaction by the individual, e.g., by stiffening a tissue region, or applying pressure to a tissue region, or electrically stimulating a tissue region to prompt the individual to change their sleep position or posture or breathing architecture and return to a sleep state more conducive to deep, restorative sleep.

Operation of the corrective action element 30 disturbs the individual in a tactile, auditory, or other sensory way sufficient to arouse the individual, thereby teaching the individual to alter their sleep position or posture and thereby return to a sleep state more conducive to deep, restorative sleep.

Desirably, operation of the corrective action element 30 will affect the individual on a lower tactile, auditory, or other sensory level that does not necessarily arouse the individual. In this arrangement, operation of the corrective action element 30 creates a sensory output having a duration or magnitude that will not necessarily awake and/or arouse the individual and/or subconsciously disturb or change or interrupt the sleep state of the individual, but nevertheless will lead to a subconscious reaction, changing the sleep position or posture, or changing an undesired physiologic state to open the airway.

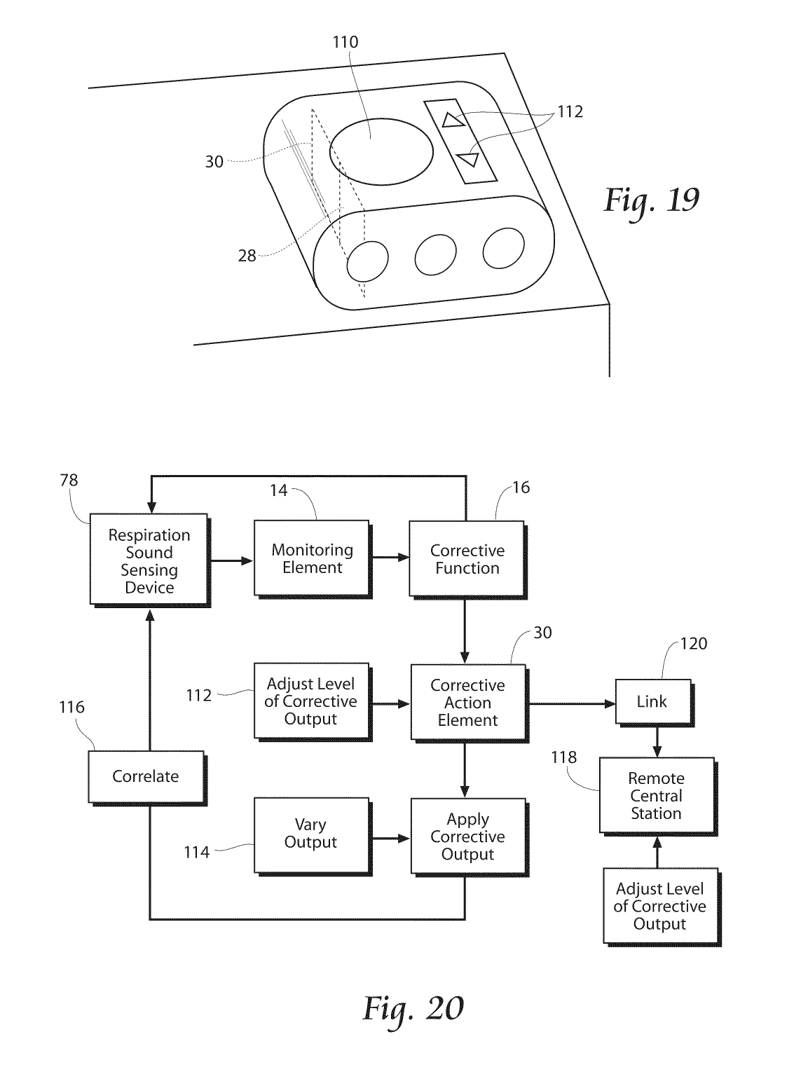

The corrective action element can include a pre-set level of corrective output. Desirably, as FIGS. 19 and 20 show, the third component 16 can includes a keypad or dial or some form of interface 112 to allow the individual or caregiver to manually set and adjust the magnitude and/or duration of corrective output from a low level to a high level. Alternatively, or in combination, the corrective action element 30 can, by pre-programmed rules 114 (see FIG. 20), automatically vary the magnitude and/or duration of corrective output in increments from low to high until the desired change in the sleeping state of the individual is sensed by the first component 12, resulting in a terminating of the alarm output. In the arrangement, the processing element serves to ramp the magnitude and/or duration of corrective output according to a prescribed steps or increments until a prescribed maximum magnitude and/or duration is reached.

The corrective action element 30 can include a correlation function 116 that compares the level and/or duration of the corrective output with termination of the alarm output and iteratively adjusts the level and/or duration of subsequent application of the corrective output according to the correlation. In this way, the corrective action element 30 learns and adjusts the level and/or duration of corrective output based upon the individual's sleep performance.

In an alternative embodiment (see FIG. 20), the third component 16 can be linked to a remote central station 118 by landline or internet connection link 120. Through the link 120, caregivers at the central station 118 can review the correlation between the level and/or duration of the corrective output with termination of the alarm output and remotely adjust the level and/or duration of subsequent application of the corrective output according to the correlation.

B. Active Corrective Action

Operation of the corrective action element can affect the individual's sleep position or posture by actively altering the configuration of sleep surface itself. Various representative examples of active corrective action devices are described below.

1. Controllable Sleep Surfaces

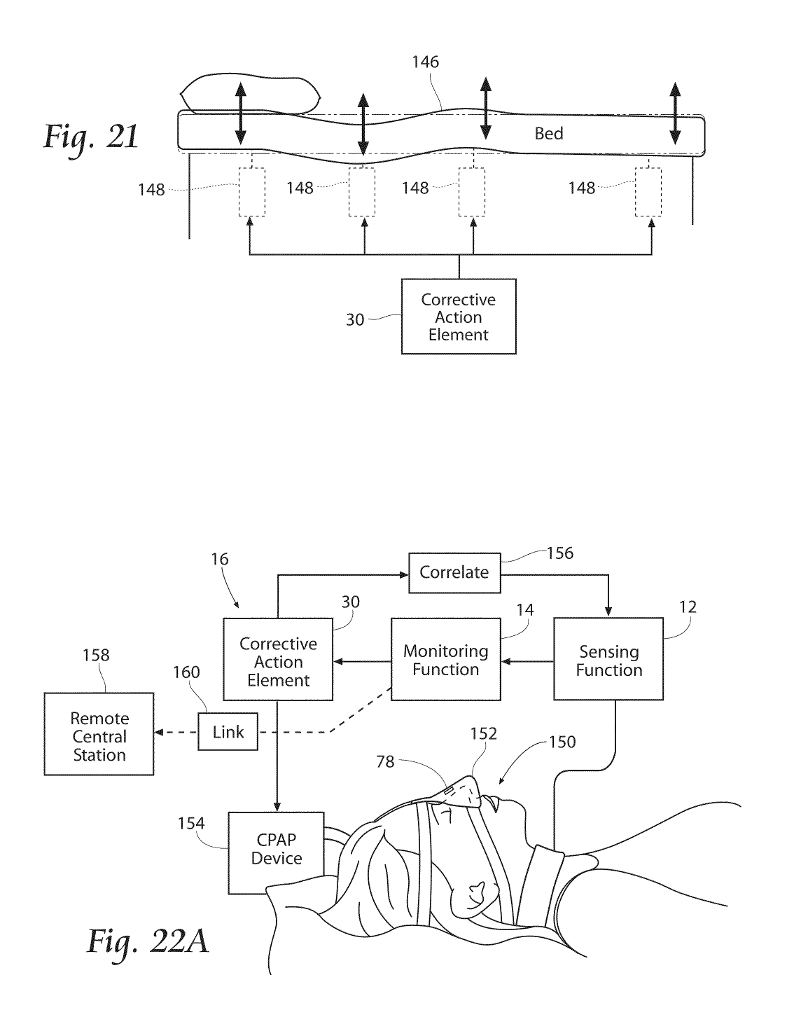

FIG. 21 shows a representative embodiment of an active corrective action element controllable by the corrective action element 30 of the third component 16. In this embodiment, the element comprises a variable sleep surface 146 that can be controlled to alter the sleep position or posture of the individual.

The variable sleep surface 146 can be a mattress and/or a pillow. The sleep surface 146 includes actuators 148 that articulate the sleep surface 146 to encourage a desired sleep posture. For example, as shown in FIG. 18, the sleep surface 146 can be pivoted to roll a sleeping individual from their back onto their side. The form, fit, and function of actuators can vary. They can be mechanical lifters or pneumatic lifters.

Furthermore, the sleep surface 146 itself need not be physically articulated, but instead the comfort of different regions of the sleep surface can be pneumatically varied (from hard to soft, or from hot to cold, or from stationary to vibrating, using, e.g., a pneumatic SLEEP COMFORT™ Mattress and the like) to encourage the individual to shift sleep positions until a comfortable sleep surface and posture are found.

A variable sleep surface 130 as just described can be readily integrated into an overall therapeutic system, like that shown FIGS. 1 and 2, which is sleep position sensitive and/or like that shown in FIG. 14A, which is sensitive to the architecture of an individual's sleeping sounds. In these arrangements, the second component 14 monitors and processes the sensed sleep position and/or sleep sound architecture and generates an alarm output if the monitored sleep position or sleep sound architecture does not conform to the “best” or desired benchmark. The third component 16 receives the alarm output and generates signals to control the configuration or the sleep surface 146 until one or more benchmark conditions return.

2. Controllable External Sleep Aids

FIG. 22A shows another representative embodiment of an active corrective action element controllable by the corrective action element 30 of the third component 16. In this embodiment, the element 30 comprises serves to control an external sleep aid 150, such as a positive airway pressure (PAP) machine, or another device associated with the individual to control physiologic conditions conducive to deep, restorative sleep.

a. PAP/CPAP

As shown in FIG. 22A, the element 30 is coupled to a positive airway pressure system, which in the illustrated embodiment is a continuous positive airway pressure (CPAP) system. The CPAP system includes delivery device 152, such as a nasal pillow, nose mask or full-face mask, and a machine 154 that delivers a stream of compressed air to the delivery device at a prescribed pressure, which is also called the titrated pressure. The intent of positive airway pressure, e.g., CPAP is to splint the airway (keeping it open under air pressure) so that unobstructed breathing becomes possible, reducing and/or preventing snoring, apneas, and hypopneas.

The necessary titrated pressure applied is usually determined by a sleep physician after review of a study supervised by a sleep technician during an overnight study (polysomnography) in a sleep laboratory. The titrated pressure is the pressure of air at which most (if not all) apneas and hypopneas have been prevented, and it is usually measured in centimeters of water (cm H₂O). The pressure required by most patients with sleep apnea ranges between 6 and 14 cm H₂O. A typical CPAP machine 154 can deliver pressures between 4 and 20 cm H₂O. More specialized units can deliver pressures up to or 30 cm H₂O and some can automatically titrate pressure based upon various inputs.

It has been observed that for most individuals using CPAP, the optimal titrated pressure is significantly higher when the individual rest in a supine (on the back) position than in a lateral (on the side) position. In one study, the mean optimal titrated pressure for an individual resting in a supine position was observed to be 10.00+/−2.20 cm H₂O, whereas the mean optimal titrated pressure for an individual resting in a lateral position was observed to be 7.61+/−2.69 cm H₂O.

The data suggests that lower titrated pressure is warranted when an individual is in the “best” or desired sleep position.

An overall therapeutic system, like that shown in FIGS. 1 and 2, which is sleep position sensitive can be readily integrated in a positive airway pressure system, e.g., CPAP. In this arrangement, the sleep position of the individual undergoing positive airway pressure therapy is sensed by the first component 12 and monitored by the second component 14. The processing element 20 of the second component 14 is pre-programmed to differentiate when the individual's sleep position(s) conform to the “best” or desired sleep position(s) and when they do not. This functionality has been earlier discussed in detail.

In this arrangement, the corrective action element 30 of the third component 16 may be programmed to respond to the alarm output by affecting an operating condition of the machine 154 to influence or alter the physical and/or physiologic sleep condition of the individual to return the individual to a physical and/or physiologic sleep condition that correlates to a desired physical and/or physiologic sleep condition. For example, the corrective action element 30 can affect an operating condition of the machine 154 by initiating an increase in titrated pressure supplied by the machine 154, e.g., by ramping the titrated pressure upward according to a prescribed steps or increments until a prescribed optimal pressure for that sleep position is reached. This response is consistent with the need for higher titrated pressure when the individual rests in an undesired sleep position. The change in titrated pressure will also be sensed by the individual undergoing positive airway pressure therapy like CPAP, and will encourage sleep position change.

Also, in this arrangement, the processing device of the third component 16 may be programmed to respond to a transition from an undesired sleep position to a desired sleep position, by initiating a decrease in titrated pressure, e.g., by ramping the titrated pressure downward according to a prescribed steps or increments until a prescribed optimal pressure for that sleep position is reached. In this way, the automatic positive pressure machine can take into account the sleep position of the individual.

The corrective action element 30 can include a correlation function 156 that compares the titrated pressure with termination of the alarm output and iteratively adjusts the maximum pressure according to the correlation. In this way, the corrective action element 30 learns and can optimize the titration pressure based upon the individual's sleep performance.

In this way, the system makes possible the controlled delivery of optimal positive airway pressure in a manner that is correlated with the sleep position of the individual, enhancing the likelihood of CPAP therapy compliance.

In this arrangement, the position-sensitive element may be as previously described, worn by the individual or externally located. The position-sensitive element may also be integrated with the positive airway pressure delivery device, such as a nasal pillow, nose mask or full-face mask.

Alternatively, or in combination with actively affecting an operating condition of the positive pressure generator of the machine, the corrective action element 30 of the third component 16 may be programmed to respond to the alarm output by generating at least one sensory or physiologic disturbance to influence or alter the physical and/or physiologic sleep condition of the individual to return the individual to a physical and/or physiologic sleep condition that correlates to a desired physical and/or physiologic sleep condition. For example, operation of the corrective action element 30 can affect the individual in a tactile, auditory, or other sensory way (e.g., by use of a buzzer or one or more flashing lights) sufficient to arouse the individual, thereby teaching or conditioning the individual to alter their sleep position or posture and thereby return to a sleep state more conducive to deep, restorative sleep. Alternatively, and more preferably, operation of the corrective action element 30 can affect the individual on a lower tactile, auditory, or other sensory level that does not awake and/or arouse the individual and/or subconsciously disturb or change or interrupt the sleep state of the individual. In this more preferred arrangement, operation of the corrective action element creates a sensory output having a duration or magnitude that will not necessarily awake and/or arouse the individual and/or subconsciously disturb or change or interrupt the sleep state of the individual, but nevertheless will lead to a subconscious reaction, changing the sleep position or posture or muscle tension in the upper airway. Alternatively, operation of the corrective action element 30 can affect the individual's sleep position or posture by actively altering the orientation or configuration of sleep surface itself. In this arrangement, the corrective action function 30 can articulate or inflate a pillow or a mattress to alter the sleep position or posture of the individual.

Like the position-sensitive element, the corrective action element may, be as previously described, worn by the individual or externally located. Like the position-sensitive element, the corrective action element 30 may also be integrated with the positive airway pressure delivery device, such as a nasal pillow, nose mask or full-face mask.

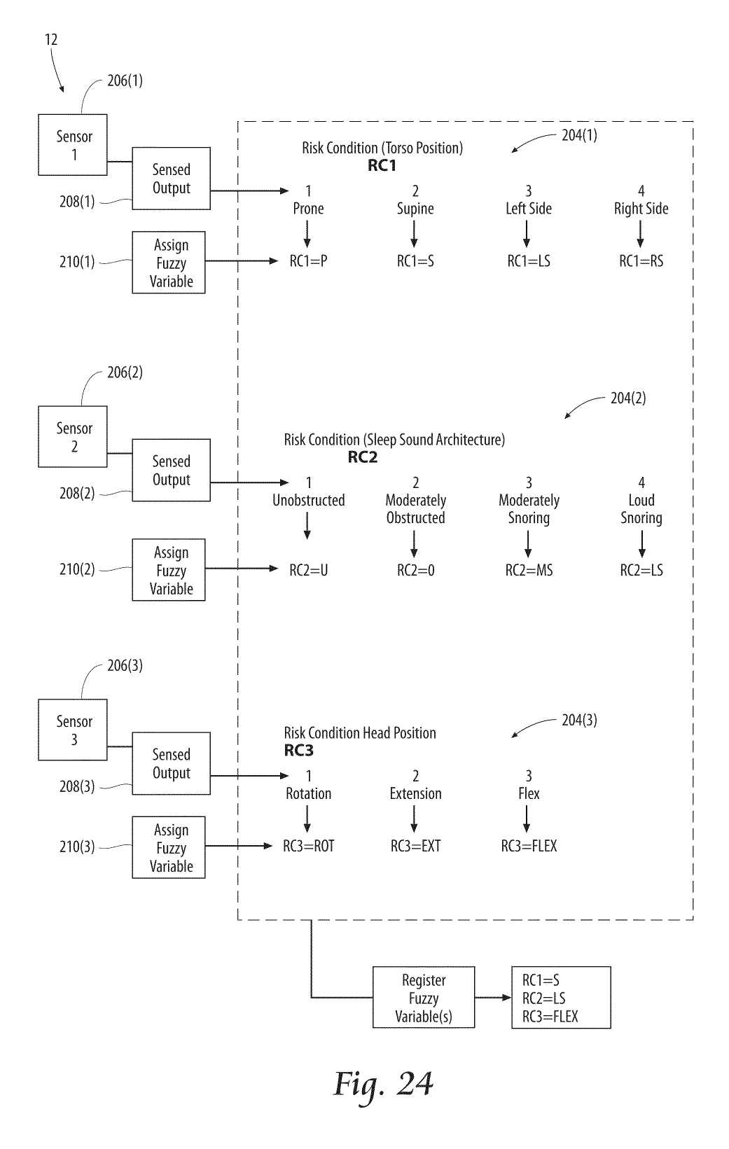

Furthermore, an overall therapeutic system, like that shown in FIG. 14, which is sensitive to the architecture of an individual's sleeping sounds, can be readily integrated in a positive airway pressure system such as CPAP. In this arrangement, the sound sensitive element 78 (e.g., one or more microphones) can be integrated into positive airway pressure delivery device (as shown in FIG. 24), such as a nasal pillow, nose mask or full-face mask, or placed bed side in the manner previously described. Coupled to the first component 12, the microphone 78 detects the sleep sound architecture of the individual. The second component 14 monitors and processes the sleep sound architecture and generates an alarm output if the monitored sleep sound architecture does not conform to the “best” or desired benchmark. The third component 16 receives the alarm output and generates signals to either affect an operating condition of the machine or generate at least one sensory or physiologic disturbance to influence or alter the physical and/or physiologic sleep condition of the individual to return the individual to a physical and/or physiologic sleep condition that correlates to a desired physical and/or physiologic sleep condition, or both. As before described, operation of the machine can be affected by initiating an increase in titrated pressure, e.g., by ramping the titrated pressure upward according to a prescribed steps or increments until a prescribed maximum optimal pressure for that sleep position is reached. The increase in titrated pressure will be sensed by the individual undergoing positive airway pressure therapy, and will encourage a modification of the sleep sound architecture. Likewise, in this arrangement, the processing device of the third component 16 may be programmed to respond to a transition from an undesired sleep sound architecture to a desired sleep sound architecture, by initiating a decrease in titrated pressure, e.g., by ramping the titrated pressure downward according to a prescribed steps or increments until a prescribed minimum optimal pressure for that sleep position is reached.

The corrective action element 30 can include a correlation function that compares the titrated pressure with termination of the alarm output and iteratively adjusts the maximum pressure according to the correlation. In this way, the processing element of the corrective action element learns and can optimize the titration pressure based upon the individual's sleep performance.

In this way, the system makes possible the controlled delivery of optimal positive airway pressure pressure in a manner that is correlated with the sleep sound architecture of the individual, enhancing the likelihood of CPAP therapy compliance.

Communication between the processing element of the third component 16 and the controller of the positive airway pressure machine can be established by interconnecting cables or by wireless signals, such as infrared or radio frequency waves including Blue Tooth™ technology.

As can by now be appreciated, any form of a sensing component that has been previously described, which senses one or more physical and/or physiologic sleep conditions of an individual, can be integrated with a positive airway pressure system, like CPAP. The sensing component generates a sleep condition output indicative of the physical and/or physiologic sleep condition of the individual. A companion monitoring component communicates with the sensing component to compare the sleep condition output with one or more benchmark conditions that correlate to a desired sleep physical and/or physiologic condition. The monitoring component generates an alarm output when a desired physical and/or physiologic sleep condition is absent. A companion corrective action component communicates with the monitoring component and includes a corrective action element that, in response to the alarm output, either affects an operating condition of the positive pressure generator and/or generates at least one sensory or physiologic disturbance and/or alters an orientation or configuration of a sleep surface, to influence or alter the physical and/or physiologic sleep condition of the individual to return the individual to a physical and/or physiologic sleep condition that correlates to a desired physical and/or physiologic sleep condition.

In an alternative embodiment (shown in FIG. 22A), the integrated system comprising a positive airway pressure system and a position and/or sound sensing and monitoring system can be linked to a remote central station 158 by landline or internet connection link 160. Through the link 160, caregivers at the central station 158 can monitor the delivery of titrated pressure and the individual's sleep position and/or sleep sound architecture and remotely adjust the magnitude of the titrated pressure accordingly.

b. Therapeutic Oral Appliance

Just as a sound sensitive element 504 can be integrated into an oral device in the manner shown in FIG. 22B for measuring sound energy flow from within the oral cavity, a sound sensitive element 504 can be integrated into a therapeutic oral appliance. The term “therapeutic oral appliance” means an oral appliance that fits over the upper and lower teeth and is sized and configured, to hold the tongue and/or push the lower jaw forward and serve as an alternative to CPAP therapy for the treatment of obstructive sleep apnea.

In this arrangement, the corrective action element 30 may serve to adjust the therapeutic oral appliance to extend the jaw forward to generate a larger airway.

Other sensing elements may also be incorporated into the therapeutic oral appliance alone or in combination with the sound sensitive element 504.

c. Alarm Clock/Radio

The corrective action element 30 can comprise an alarm clock/radio. In this arrangement, a sensing function senses sleep state (stage/REM) or EOG (a measure of REM sleep). The processing element 20 of the monitoring element 14 monitors the sleep cycle of the individual. The monitoring element 20 is coupled to the alarm clock/radio. The processing element 20 prevents activation of the alarm to awake the individual from sleep, until the processing element 20 indicates that the individual is at a proper point of their sleep cycle. In this way, the individual is not aroused at a point of their sleep cycle that causes them to awake tired.

### V. Therapeutic Devices, Systems and Methods Including a Learning Function

FIG. 23 shows a therapeutic system 200 that includes the components 12, 14, and 16 that serve complementary sensing, monitoring, and corrective functions.

The system 200 further includes a table 202 of one or more preselected physical and/or physiologic conditions 204 that can be considered, based upon empirical clinical data applicable to a general population of individuals experiencing sleep apnea, predictors of a potential apnea event. The conditions listed in the table 202 will also be called the Apnea Risk Conditions. FIG. 24 shows the table 202 in more detail.

Representative Apnea Risk Conditions 204 have been generally described previously in other contexts. They include, but are not limited, to (i) the torso position of the individual (supine, prone, left side, right side); (ii) the head position of the individual (rotation, flexion, extension); (iii) the architecture of breathing sounds or vibrations the individual makes while sleeping; and (iv) certain physiologic conditions of the individual that can be sensed, such as peripheral arterial tone; blood pressure; the level of oxygen in the blood (oxygen desaturation/blood saturation); chest and diaphragm effort during inhalation and expiration; EEG; EMG (electrical muscle activity); heart rate; respiration or breathing rate; arrhythmia detection; incidences of periodic cessation or interruption of breathing; sleep fragmentation; arousals; sleep state (stage/REM); EOG (measure of REM sleep); measured inhalation and exhalation airflow, or vibration of the airflow current (e.g., by use of a nasal cannula); sensed neural signals; airway resistance/flow restriction; positive airway pressure flow resistance; pharyngeal critical closing pressure Pcrit; neck tissue compression; and/or muscle tension/strain. The Risk Conditions can also include data entered by the individual indicative of medication being taken, the amount of alcohol consumed, the presence or absence of a sleep partner, and other conditions that may affect the character of the individual's sleep.

As FIG. 24 shows, the system 200 includes sensing components 206 that sense for a particular individual selected one or more of the Apnea Risk Conditions 204 listed in the table 202. The table 202 can also list the magnitude 208 of the sensed value for the sensed Apnea Risk Condition 204. In the illustrated embodiment, the table 202 also lists a dimensionless fuzzy variable 210 for at least one of the sensed Apnea Risk Conditions 204. The fuzzy variable 210 is indicative, according to pre-programmed rules, of the correlation of the sensed value to a predetermined benchmark condition. The fuzzy variable 210 is assigned by a processing function based upon a comparison of the sensed condition to the respective benchmark condition.

For example, as shown in FIG. 24, one sensed Apnea Risk Condition RC1 is the torso position of the individual. The listed sensed values are value 1 for prone; value 2 for supine; value 3 for left side; and value 4 for right side, based upon the output of a position sensor worn by the individual, as previously described. The processing function assigns a fuzzy variable 210(1) to each position value according to preprogrammed rules, e.g., 1=P; 2=S; 3=LS; and 4=RS.

As FIG. 24 also show, another sensed Apnea Risk Condition 208 RC2 is sleep sound architecture, which can be expressed as a binary number indicative of the pattern or signature of the sound energy flow, in terms of amplitude, frequency, and duration, as previously described. The processing function assigns a fuzzy variable 210(2) to the binary expression according to preprogrammed rules residing in the processing function, e.g., (U) Unobstructed; (O) Moderately Obstructed; (MS) Moderate Snoring; (LS) Loud Snoring.

As also shown in FIG. 24, another sensed Apnea Risk Condition RC3 is the head position of the individual (sensed value 1 for rotation; sensed value 2 for extension; and sensed value 3 for flex), based upon the output of a head position sensor worn by the individual, as previously described. The processing function assigns a fuzzy variable 210(3) to each position value according to preprogrammed rules, e.g., 1=ROT; 2=EXT; 3=FLEX.

In the illustrated embodiment, the table 202 registers the fuzzy variables 210(1), 210(2), and 210(3) based upon the sensed outputs 208(1), 208(2), and 208(3), that indicate the individual is laying on their back (RC1=S) with their head in flexure (RC3=FLEX), and further snoring loudly (RC2=LS).

In the illustrated embodiment shown in FIG. 23, the system 200 also includes a table 212 of preselected corrective actions 214 that, for a general population of individuals experiencing sleep apnea, can influence or alter the individual's sleep position, sleep sound architecture, or sleep state. Representative corrective actions 214 have been generally described previously in other contexts. Representative corrective actions 214 can include the activation of an apnea therapy device 216, such as a positive airway pressure system, neck brace collar, a scaffold device in a tongue or floor of the mouth, a tongue suspension device, a genioglossus or nerve or muscle stimulation device, a palate, pharynx, tongue, or other tissue stiffening or reshaping device, a head positioning device, a neck positioning device, a mandible positioning device, or a torso positioning device, or combinations thereof. The corrective action 214 can also include a device 218 that generates a sensory and/or physiologic disturbance to interrupt an individual sleep state or arouse or awaken an individual, such as by generating an audible tone, another sensory stimulation such as smell or vibration, or by stimulating nerve or muscle, or combinations thereof. The system 200 includes one or more of the corrective action devices 216 or 218 capable of performing selected one or more of the corrective actions 214 listed in the table 212.

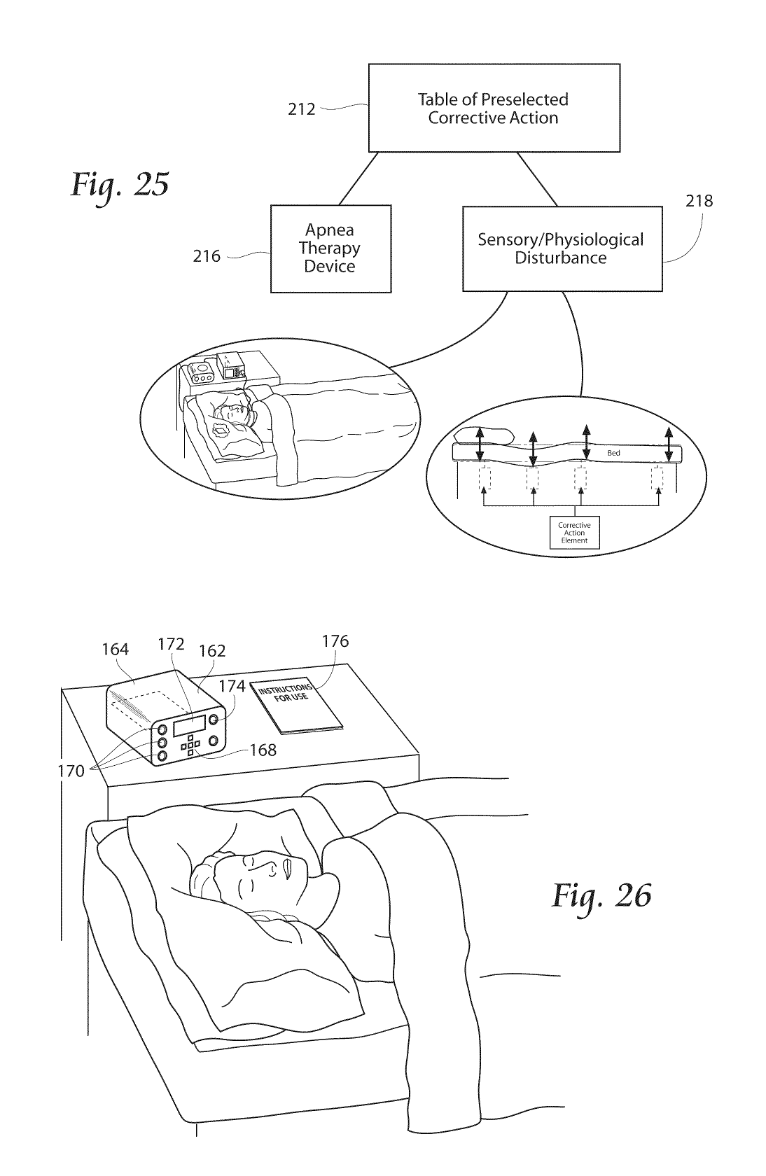

As shown in more detail in FIG. 25, the system 200 includes a corrective action device 218 that comprises a speaker or earphone 110 through which an audible sound or vibration is generated (like that shown in FIG. 1). Desirably, the corrective action output of the corrective action device 218 can be varied, from a low volume state to a high volume state. The audible sound can thereby be titrated to interrupt the individual's sleep state without necessarily awaking the individual, which is a desirable outcome for reasons previously explained.

In the embodiment shown in FIG. 22, the system 200 also includes another corrective action device 218 that comprises a variable sleep surface 146, like that shown in FIG. 21. Desirably, the corrective action output of the sleep surface 146 can be varied, e.g., by pivoting to roll the individual from their back to their side or by changing the sleep surface from soft to hard, making it more uncomfortable, or by causing the sleep surface to vibrate. The varying outputs provide alternative means to encourage an individual to arouse and shift their sleep position, again without necessarily awaking the individual.

As FIG. 23 shows, the system 200 further includes a continuous monitoring function 220. The monitoring function 220 periodically derives information from one or more of the sensing devices associated with the individual. The monitoring function 220 includes a processing function 222 that processes the sensed information according to pre-programmed rules to detect the presence or absence of an apnea sleep event.

The onset of an apnea sleep event can be detected in various ways. For example, a sensing device can sense oxygen level in the individual's blood, and the processing function 222 can analyze changes in the blood oxygen level to detect oxygenation desaturation that is indicative of an apnea sleep event. Alternatively, or in combination, the sensing device can sense pauses or cessation of breathing, and the processing function 222 can correlate this data to an Apnea-Hypopnea Index (AHI). This is the embodiment illustrated in FIG. 23. The magnitude of the AHI assesses the severity of sleep apnea based on the total number of complete cessations (apnea) and partial obstructions (hypopnea) of breathing occurring per hour of sleep. Typically, the pauses in breathing must last for several seconds and are associated with a decrease in oxygenation of the blood. In general, the AHI can be used to classify the severity of the apnea event from, e.g., not existent AHI=0-5); mild (AHI=5-15); moderate (AHI=15-30), and severe (AHI is greater than 3%). Other indications of an apnea event that can be sensed and processed by the processing element 222 include diaphragm effort, EEG, or indications of the Apnea Risk Conditions described above.

The monitoring function 220 periodically receives as input the sensed apnea sleep event information, which the processing function 222 analyzes according to pre-programmed rules to yield an output, e.g., the AHI as shown in FIG. 23. The output indicates whether or not an apnea sleep event exists at that point in time. If the AHI value—AHI=(N)—falls below a predefined threshold value (e.g., less than 5), the monitoring function 220 processes the next periodically sensed information. If the output indicates that an apnea sleep event is occurring (e.g., the AHI is greater than 5), the monitoring function 222 outputs the corresponding AHI value as an apnea event alert 224. The apnea event alert 224 can be expressed as an AHI integer value ranging from −15 (mild), 15-30 (moderate), and greater than 30 (severe).

The system 200 includes a learning function 226. The learning function 226 serves two purposes. First, the learning function 226 serves to identify from the table 202 the particular sensed physical and/or physiologic conditions that are best indicative of why the sleep-related problem is occurring for that particular individual. Second, the learning function 226 serves to identify the particular corrective function or functions that are best suited for that particular individual to correct the problem.

In serving the first purpose, the learning function 226 receives the AHI alert value 224. The learning function 226 looks to the table 202 of sensed Apnea Risk Conditions 204 and registers the respective fuzzy variables 210 that are associated with the generation of the AHI alert value 224. According to preprogrammed rules, the learning function 226 selects the one or more sensed Apnea Risk Conditions 204 having the fuzzy variables 210 that best indicate why an actual apnea event has occur.

For example, using fuzzy logic principles, a logic table 228 residing in the learning function 226 can dictate the identification of the risk condition or conditions best indicative of the sleep related problem that the AHI alert value 224 represents, according to a pre-programmed rule expressed generally as IF X AND Y THEN Z.

For example, in the context of FIG. 23, a pre-programmed rule can read: IF RC1=S AND AHI Value=15 to 30 THEN Select Apnea Risk Condition RC1: Torso Position. A pre-programmed second rule may also apply to further guide and confirm the selection: e.g., IF RC2=LS AND AHI Value=15 to 30 THEN Select Apnea Risk Condition RC1: Torso Position. Another pre-programmed rule may lead to a different selection Z based upon the nature of the X and Y conditions of the rule: e.g., IF RC3=FLEX and AHI Value=5 to 15 THEN Select Apnea Risk Condition RC3: Head Position.

The pre-programmed IF X AND Y THEN Z rules may also change the selection based upon the magnitude of the AHI Value. For example, the rule may guide the selection of Z (Select Apnea Risk Condition RC3: Head Position) whenever the AHI Value is 5-10, regardless of torso position, and may guide the selection of Z (Select Apnea Risk Condition RC1: Torso Position) whenever the AHI Value is >10.

By understanding and characterizing physiologic events associated with sleep apnea events using clinical knowledge and experience (both in general and as are ascertained for the specific individual through sleep testing or use of diagnostic tools), the learning function 226 can be systematically developed using fuzzy rules, which describe the principles of the regulation of sleep conditions for the individual in terms of the relationship between inputs (the apnea risk conditions sensed) and outputs (the presence or absence of an apnea sleep event).

In serving the second purpose, the learning function 226 also looks to table 212 of corrective actions 214. According to preprogrammed rules, based upon the selected Apnea Risk Condition or Conditions 204 (as just described) (from the table 202), the learning function 226 selects one or more corrective actions 214.

For example, using fuzzy logic principles, a logic table 230 residing in the learning function 226 can dictate a selection of a corrective action according to a preprogrammed rule IF X AND Y THEN Z. For example, in the context of FIG. 23, the pre-programmed rule can read: IF RC1=S AND AHI Value >5) THEN Activate the Corrective Action Device 216 that comprises the speaker 110.

A second pre-programmed rule may also apply to guide a different selection: e.g., IF RC2=MS or LS AND AHI Value >15 THEN Activate Corrective Action Device 216 that comprises a Variable Sleep Surface 146.

Another pre-programmed rule may also dictate taking no corrective action, due to the nature of the sensed conditions. For example, the pre-programmed rule can read IF RC2=MS AND AHI Value <5) THEN Take No Corrective Action.

Using fuzzy logic principles, the learning function 226 can systematically select among one or more corrective actions, or chose to take no corrective action, depending upon the relationship of the sensed conditions. As before stated, by understanding and characterizing physiologic events associated with sleep apnea using clinical knowledge and experience (both in general and customized for the individual), the learning function 226 can be developed using fuzzy rules, which describe the principles of the function's regulation of sleep conditions in terms of the relationship between inputs (the sensed apnea risk conditions) and outputs (the correction action to be taken).

The learning function 226 continuously monitors and assesses the best-indicated physical and/or physiologic conditions and corrective functions for that particular individual. The learning function 226 further includes a customization function 232 that optimizes the selected corrective action for the particular individual.

More particularly, based upon pre-programmed rules, the customization function 232 creates a rule structure or matrix 234 listing corrective parameters optimized for the individual for the particular corrective action that is selected. The rule matrix 234, once developed, guides the nature of corrective action that will, for the individual, provide the “best” corrective result, in terms of moderating the duration of the apnea event with the avoidance or minimization of arousal, physiologic reaction, and other undesirable corrective effects upon the individual.

In a representative embodiment, the rule matrix 234 is developed by the customization function 232 by titrating the magnitude of the corrective action in real time and observing consequent changes in magnitudes of the AHI Values and how quickly these changes in the AHI Values occur (ΔAHI), until the AHI Value returns to a prescribed desirable magnitude indicative of the absence of an apnea event, e.g., AHI<5. The rule matrix 234 can be developed by also taking into account arousal of the individual by concurrently sensing the individual sleep state as corrective action is being titrated.

A representative rule matrix 234 developed by the customization function 232 can take the following form:

### Representative Rule Matrix

| | AHI Value (X) | | |
|---|---|---|---|
| | 0 to 5 | 5-15 | 15+ |
| Effect (Y) | None | Mild | Moderate to Severe |
| D | C = 0 (Z) | C = +1 (Z) | C = +1 (Z) |
| AHI Value Getting | | | |
| Better (Decreasing) | | | |
| 0 | C = 0 (Z) | C = +2 (Z) | C = +2 (Z) |
| No Change in AHI | | | |
| Value | | | |
| I | C = +1 (Z) | C = +3 (Z) | C = +3 (Z) |
| AHI Value Getting | | | |
| Worse (Increasing) | | | |

Where: C is the magnitude of the corrective effect; 0 indicates no change in the corrective action magnitude; and +1, +2, and +3 indicate a titration of the corrective action magnitude by 1, 2, and 3 units, respectively. The optimization function 230 may impose an absolute maximum value for the correct action magnitude (e.g. C=5) that cannot be exceeded.

The rule matrix 234 expresses a set of IF X AND Y THEN Z rules optimized by the customization function 232 for the individual. For example, according to the rules expressed in the rule matrix 234 shown above, IF AHI Value=5-15 AND AHI Is Getting Worse (i.e., increasing in value) THEN Increase the Corrective Effect by 3 units. The rule matrix 332 expresses different corrective action as AHI and ΔAHI change. For example, according to the rules expressed in the rule matrix 330, IF AHI Value=15+ AND AHI is Showing No Change THEN Increase the Corrective Effect by 2 units.

The rules expresses for the individual, based upon the current status of the AHI value (in terms of AHI and Δ AHI as measured in real time), the magnitude of the corrective effect that the customization function 232 has selected to provide the “best” corrective result, in terms of moderating the duration of the apnea event with the avoidance or minimization of arousal, physiologic reaction, and other undesirable corrective effects upon the individual.

In the same fashion, the customization function 232 creates a rule matrix for each form of corrective action that can be selected. The learning function 220 thus creates for the individual a validated rule structure 236 comprising a set of pre-programmed rules that are customized for the individual based upon the nature of the apnea event, which in the illustrated embodiment, is expressed in terms of an AHI Value. Different degrees of apnea events (leading to different AHI Values) may, according to the validated rule structure 236, call for different types of corrective response or responses, and, within a given corrective response, different degrees of corrective effects, depending upon what has been demonstrated by previous responses to work best for the individual.

Once the learning function 226 establishes the validated rule structure 236 applicable to the array of corrective actions available, the system 200 may proceed directly from the monitoring function 222 to the validated rule structure 236 in responding to a given apnea event. The system 200 may, however, periodically chose not to correct a given apnea event according to the validated rule structure 236, to assess whether the monitoring function 220 is generating a false positive output.

Desirably, the system 200 periodically performs a re-validation function 238, responding to a given apnea event by calling up the learning function 226 to process and analyze the event, select the corrective action, and develop a new validated rule structure 236. The re-evaluation function 238 adapts to changes in the individual's responses to the corrective action over time. Also, if the individual does not respond to a given validated rule matrix 236, the system 200 automatically calls up the learning function 226 to develop a new rule matrix 234. In this way, the system 200 continuously learns, adjusts, adapts, and optimizes the validated rule structure 236 to the individual over time.

In this way, the system 200 differs significantly from a conventional sleep study, which is performed during a discrete period of time (typically one night). The system 200, unlike a conventional sleep study, continuously monitors the individual over a prolonged period of time (night-after-night) and continuously learns, adjusts, adapts, and optimizes itself to the individual on a day-by-day basis.

As described, the system 200 tailors itself to the individual over time with corrective action optimization achieve the best (optimum) sleep effectiveness therapy. The system 200 serves to minimize undesirable aspects of OSA including Apnea-Hypopnea events; snoring and vibration; obstructive respiratory flow; arousals and disruption and sleep fragmentation; heart stress; the amount and amplitude of corrective action. The system 200 achieves these desirable outcomes by making it possible to (i) minimize and titrate corrective action, aiming for the lowest level of corrective action for effect; (ii) employ combination of corrective actions at lower thresholds; (iii) vary corrective actions; (iv) test to validate if corrective actions are still necessary; (v) test to see if no action can be taken; (vi) learn when should action be taken; (vii) test to see if corrective action threshold can be reduced; (viii) test to see if corrective action should shift (type, amplitude, frequency, delay) for better response, or use a larger amplitude burst pulse with delay versus ongoing vibration; (ix) determine best location to apply corrective action (location of buzzer on the body, direction of sound, etc.); (x) teach with two or more different corrective actions (one or more of which is active and may cause arousal and one or more which are “passive” and do not cause sleep disruptions), to bring about an associative learning, so that, when the “active” is removed after some time, the passive still causes the individual to modify their position. Further, the system 200 achieves desirable sleep therapy outcomes by making it possible to (i) optimize the parameter threshold prior to applying the corrective action; (ii) determine when to act, how to act and when not to act for the individual; (iii) modify and adjust type, amplitude, ramp and frequency of corrective action for best result; (iv) modify corrective action as the individual changes, adapts and is conditioned overtime, achieving adaptive corrective action; and (v) optimize timing of corrective action. The system 200 recognizes that typical sleep studies are discrete, and that the system 200 is well adapted to the collection data over long periods of time that give a better picture of the individuals sleep patterns and what inputs lead to disruptive sleep.

The system 200 also makes it possible to consider and integrate into a sleep therapy platform a diversity of inputs that may effect/delay corrective and action, such as sleep stage; time of night; time from onset of sleep; objective measures of the individual's “tiredness” (lack of sleep, alcohol or other depressants, etc., and modify corrective action accordingly. The system 200 makes it possible to take into account the disruption to the sleep partner and design “manual shut-offs” into the device by the sleep partner and otherwise allow for personal programming of parameters and resting of the system 200.

The system 200 makes it possible for a user to input “human condition” data, or to automatically register such human condition data, relating to the physiology and environment of the individual undergoing treatment so that the system 200 can integrate these data in assessing their effect of sleep quality. Such human condition data can include weight, neck size, pillow type, medications, alcohol consumption, stress, happiness, sleepiness, pre-sleep activities, room temperature, and ambient noise.

As described, the system 200 selects, in response to an alarm output, a corrective action output or a combination of correction action outputs to influence or alter the physical and/or physiologic sleep condition of the individual, to return the individual to a physical and/or physiologic sleep condition that correlates to a desired physical and/or physiologic sleep condition. The learning function of the system 200 also iteratively adjusts the selection of the corrective action output or outputs according to the sensed physical and/or physiologic sleep condition of the individual, to optimize the return of the individual to a physical and/or physiologic sleep condition that correlates to a desired physical and/or physiologic sleep condition. A sleep condition can include a sleeping position and/or the architecture of sounds or vibrations during breathing and/or a physiologic condition of the individual and/or a predictor, presence, absence, or onset of an apnea sleep or snoring event. Desirably, the learning function iteratively adjusts the selection of the corrective action output or outputs to minimize arousal and/or disruption of the sleep state. The learning function can iteratively adjust the selection of the corrective action output or outputs according to the individual's response to the corrective action output over time, as the individual learns over time to control and/or improve their physical and/or physiologic sleep condition and maintain a desired physical and/or physiologic sleep condition. Thus, as the individual's physical and/or physiologic sleep conditions are controlled and/or improved by operation of the learning function, the learning function responds by adjusting the magnitude and/or type of the corrective action output accordingly.

The selected corrective action output of the system 200 can include at least one sensory or physiologic disturbance, and the system 200 can serve to iteratively adjust the selection of the corrective action output by titrating the magnitude and/or type of the sensory or physiologic disturbance to minimize arousal and/or disruption of the sleep state.

In another arrangement, or in combination with a sensory or physiologic disturbance, the selected corrective action output can adjust a variable sleep surface, and the system 200 can serve to vary the adjustment of the variable sleep surface.

### VI. Diagnostic Home Screening Device

Obstructive Sleep Apnea (OSA) is estimated to have an incidence of twenty-four percent (24%) in men and nine percent (9%) in women. Some researchers believe that up to ninety-three percent (93%) of women and eighty-two percent (82%) of men with moderate to severe OSA remain undiagnosed.

Sleep studies involving full polysomnography are prescribed by a physician and are expensive. They involve an inconvenient overnight stay in a sleep clinic. Results are complex and voluminous and must be interpreted by a medical professional trained in sleep medicine.

Home diagnostic devices are currently available and are becoming well recognized as viable alternatives to full polysomnographs. They also require a prescription and while less expensive than a full polysomnography, are still beyond the financial means of many people. They remain complex enough to require training and a learning curve to properly use the device and results must be interpreted by a doctor or trained sleep professional.

Present means of gathering information on the sleeper's breathing depend heavily on what is said by the person's sleeping partner. This information is not always available, or totally objective or reliable. The perception by the sleeping partner about how much snoring, how loud and whether breathing stops can be inaccurate and is often skewed by a variety of factors.

FIG. 26 shows a diagnostic home screening device 162 for individuals experiencing OSA, snoring, or other forms of sleep obstructive breathing. The device 162 is intended to be a single use or disposable due to low cost. The device includes sleep event sensing functions and associated processing functions that monitor and differentiate among conditions of light or no snoring, in which there is no need for clinical concern; heavy snoring, in which making certain sleeping changes may reduce the severity; and light, moderate or severe OSA, in which consultation with a sleep professional is indicated.

In one embodiment, the device 162 includes a housing 164 sized and configured to be hand-held, or placed bed-side, or worn on the body. In this respect, the device 164 can be as small and compact as a conventional cell phone or MP3 player and be sold through point of purchase sites or by mail order through television, radio, or internet commercials or “infomercials.”

The housing 164 desirably houses a processing element 166, which can comprise a microprocessor implemented on an integrated circuit board. The device also includes an output panel 168. In its simplest form, the output panel 168 can take the form of one or more lighted indicators 170, e.g., one to indicating an “on” state and one or more others to communicate an output diagnosis to the user. For example, a single lighted indication glows green when the diagnosis is light or no snoring; glow yellow when the diagnosis is simple snoring; and glows red when the diagnosis correlates with light, moderate or severe OSA or snoring.

In an alternative embodiment, the output panel 168 comprises a display screen 172, e.g., a liquid crystal display, which presents the diagnoses in words or a numeric display that refers to written instructions for interpretation. In this respect, the device 162 is as simple and easy to interpret as a home pregnancy test.

The microprocessor of the processing element 166 includes the sleep event sensing functions and associated processing functions. These, e.g., are found in embedded code, which expresses the pre-programmed rules or algorithms under which sleep events are sensed and processed, as well as the pre-programmed rules or algorithms that govern operation of the output panel.

The device 162 is desirably battery powered by use of a single use or rechargeable, standard industry-standard primary battery.

Alternatively, the sleep event sensing functions and associated processing functions of the microprocessor can be incorporated into a program that can be installed for use on an external computer or personal computer, or as an “app” on a mobile computing device (e.g., an I-Pad™ Device) or phone (e.g., an I-Phone™ Device).

The device 162 includes one or more sound-sensitive elements 174 incorporated within the housing 164 and coupled to the microprocessor of the processing element 166. Alternatively, the sound sensitive element 174 may be sized and configured to be used separate from the housing 164, e.g., placed on the body, near the body, suspended from the body, near the mouth, near the larynx (e.g., fixed with adhesive under the chin).

The sound sensitive element 174 can comprise, e.g., at least one conventional sound sensor, which is also generally referred to as a “microphone.” Various types of microphones can be used, e.g., dynamic, electrostatic, or piezoelectric. Desirably, the sound sensitive element includes an electrostatic type (condenser) microphone, for reasons earlier discussed. The sound-sensitive element measures the sound energy flow of respiration sounds made by the individual during sleep, expressed in terms of sound pressure (unit: Pa), and/or sound frequency, and/or sound frequency patterns. As before described (and as shown in FIG. 16), the architecture of a respiratory sleeping sound includes an amplitude component 80, a frequency component 82, and a duration component 84.

In use, the device 162 is purchased at a reasonable cost by an individual, e.g., as a non-prescription, over-the-counter item in a drug store. The device includes instructions for use 176. The instructions 176 direct the individual to place the device 162 bed side, turn it on (a power button is provided for this purpose), get into bed, and go to sleep. No devices need to be attached to the sleeping individual and the screening takes place in the familiar surroundings of the individual's home and own bed. During sleep in familiar surroundings, the device senses and monitors the individual's sleep sound architecture.

Desirably, the event sensing function delays its activation until after a pre-established or pre-set time period within the sleep cycle. This allows enough time for the individual to reach the desired sleep state before the sensing and processing functions are enabled.

The sleep event sensing functions of the device register the sleep sound architecture of the individual through the sound-sensitive element(s). The processing functions analyze the sleep sound architecture in terms of its amplitude, and/or frequency, and/or duration according to preprogrammed rules or other digital signal processing algorithms.

The pre-programmed rules of the processing functions of the device 162 can incorporate data from several large groups of sleep studies in terms of correlation of sensed amplitudes, frequencies, and durations to diagnostic outcomes and/or the site of the airway obstruction. Alternatively, the pre-programmed rules can incorporate data that correlates sleep endoscopy observations of airway obstructions with concurrent analysis of the particular respiration sounds resulting from the obstructions, using a methodology described above.

As a result of the processing, the processing element outputs a diagnosis. An appropriate output (the diagnosis) is generated for the output panel.

The diagnosis of the device determines if more expensive and advanced testing should be performed.

There are many advantages to this type of device. Screening done in the individual's own home and bed, which will be a more accurate indication of how they sleep than in a sleep clinic with electrodes and various monitors attached to the patient. Multiple sleep studies yield different results for any one individual, and the device makes possible the easy repetition of sleep performance monitoring. Certain results (for instance marginal ones) might be reason to instruct the individual to re-test on a subsequent night. The device readily allows all this.

Furthermore, information gathered by an electronic device may be more acceptable to the individual than what might be reported by a sleeping partner. The device allows sleep partner to demonstrate problem to an individual in objective terms. Results will be totally objective regarding snoring, cessation in breathing, duration of events, frequency and not skewed by personal feelings of the observer.

Many individual can take steps on their own to improve their sleep quality, and, with the device as a guide, experiment with different oral appliances, different apnea or snoring devices (e.g., a positive airway pressure system), different sleep positions, different hygiene, or different pillows. The device can be used to objectively measure the effects of these variables or other lifestyle changes such as caffeine or alcohol. For this purpose, the device can, if desired, include a function that retains the diagnoses arising from different sleep episodes conducted during different sleep conditions for comparison and correlation. For example, an individual can compare and correlate the diagnosis of one sleep episode with the diagnosis of a different episode, in which a sleep condition is altered, e.g., by a different sleep aid, a different pillow, a different mattress, and different nighttime habits (such as caffeine or alcohol consumption). The device can be integrated with a sleep position device, as previously described, to determine positional dependence.

In an alternative embodiment, the device can, if desired, include addition, more complex processing functions that output more information, e.g., the duration of various breathing or snoring events, the intensity of such events, etc. In this arrangement, the output panel can comprises a display screen which presents this information in a more detailed form.

The device may, if desired, also include further processing functions that generate an alarm output based upon the sleep sound architecture being monitored. In this arrangement, the device can serve as the first and second components of a therapeutic sound-sensitive system, and be coupled to a corrective action component, like that shown in FIG. 14, as previously discussed.

The systems and methods just described make it possible to provide an individual with a diagnostic home screening for obstructive breathing conditions. The systems and methods include at least one sound sensitive element that senses respiratory sounds made by the individual during a sleep session and that is sized and configured to be placed on or near the individual during the sleep session. The systems and methods also include a companion micro-processing element that is can be coupled to the sound sensitive element (either by hard wire or wireless connection, such as infrared or radio frequency waves including Blue Tooth™ technology) and that is sized and configured to be placed on or near the individual during the sleep session. The micro-processing element includes at least one pre-programmed digital sound processing algorithm that processes and registers respiratory sounds sensed by the sound sensitive element over a sleep session; compares the processed respiratory sounds registered during the sleep session to benchmark conditions correlated to a range of obstructive breathing conditions from slight to severe; selects, based upon the comparison, an obstructive breathing condition from the range of obstructive breathing conditions; and generates a diagnostic output indicative of the obstructive breathing condition selected by the pre-programmed digital sound processing algorithm. The systems and methods include a display element that is coupled to the micro-processor and that is sized and configured to visually present the diagnostic output in a format that is directly readable by the individual without interpretation by a doctor, trained sleep professional, and/or analysis out of the home. The systems and methods provide instructions for use that instruct the individual to place the at least one sound sensitive element on or near the individual during the sleep session while at home, to place the micro-processing element on or near the individual during the sleep session while at home, and to complete the sleep session while at home with the at least one sound sensitive element and the micro-processing element monitoring the individual's respiratory sounds. The instructions for use also instruct the individual to read the diagnostic output at home, without reliance on or intervention of a doctor, trained sleep professional, or an out on home analysis.

The instructions for use can optionally further instruct the individual to dispose of the system after the sleep session. The instructions for use can optionally further instruct the individual to re-test during a subsequent sleep session while at home.

As can be by now appreciated, the diagnostic home screening systems and methods as described are purposely suited for use in a home environment, being sized and configured to be entirely self-contained and installed within the confines of a bed-side or hand-held device, or likewise entirely residing as a program or app on a personal computer or small microprocessor-equipped device, such as an I-PAD™ Device or cell phone.

The diagnostic home screening systems and methods as described can utilize existing consumer electronics and household appliances. The diagnostic home screening systems and methods as described make possible a three tier product approach, which takes advantage of a microprocessor from existing consumer electronics that are commonly found in the home such as a PC; an Ipad™; a Kindle™; a phone; a gaming device, such as Nintendo™ or X-box™; Ipods™; other music devices; CPAP machines; home theater systems; and etc. The three tier approach comprises (1) supplying only software, which is placed on an existing consumer electronic, or (2) supplying software and a recording device, which are placed on an existing consumer electronics, and which incorporates means to transfer sound data, such as USB memory stick, Bluetooth™, HDMI™, etc (software could be on USB), or (3) supply hardware with embedded software.

### VII. Systematic Continuous Sleep Apnea Therapy

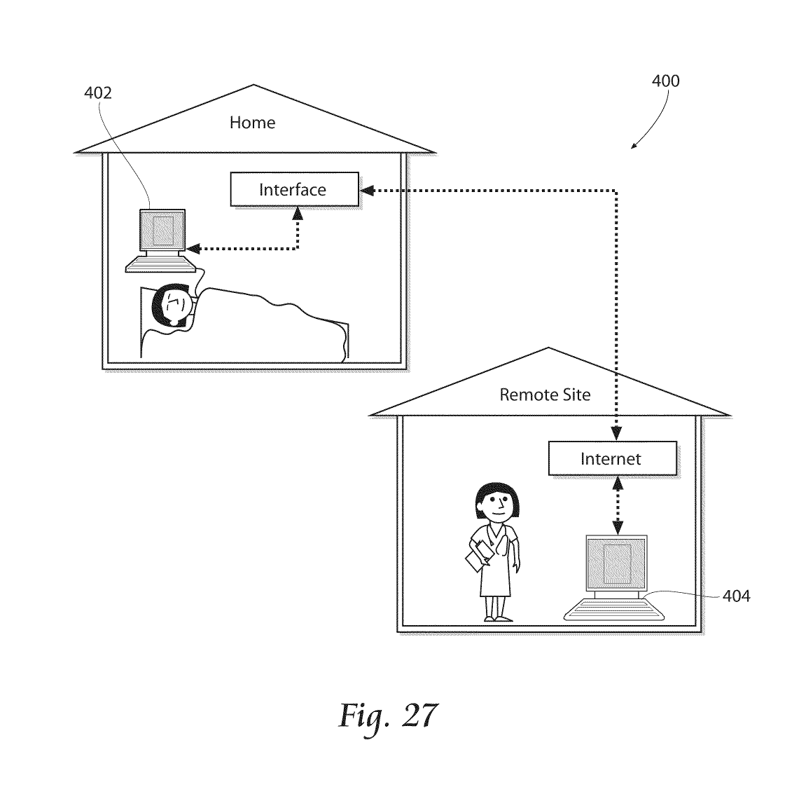

The systems and methods described herein can be integrated into a home-based continuous sleep apnea therapy system 400, as FIG. 27 shows. The system 400 continuously guides and monitors an individual experiencing OSA over a prolonged period of time (day-by-day and night-after-night). The system 400 can include a patient portal 402 that provides support and feedback and encourages an individual to take a more active and positive role in treating their OSA condition. The portal 402 is coupled by wired and wireless connections (such as infrared or radio frequency waves including Blue Tooth™ technology) to the sensing devices that allow the individual to assess their vital signs while awake, such as their blood oxygen level, heart rate, temperature, weight, respiration or breathing rate, and blood alcohol level. The patient portal 402 can incorporate the components 12, 14, and 16 so that, while asleep, these and other sensors can provide the monitoring and corrective action that leads the individual to deep restorative sleep, as described. The patient portal 402 can be linked by land line or internet connection or suitable “tele-med” link to a caregiver portal 404, which processes the information according to pre-programmed rules to provide information and historical data to them on a continuous basis that help them assess the individual's health status and control over OSA. The patient portal 402 and the caregiver portal 404 aided by an interactive touch screen, integrated audio and video, and a graphical icon-driven interface, allow the individual and caregivers to interactively respond to health assessment questions, receive educational information and motivation messages, complete surveys, etc. The patient portal 402 can also provide multimedia content to educated and assist the individual, including the ability for two-way video calls.

The system 400 transforms the treatment of OSA from the limitations of a conventional sleep study, which is performed during a discrete period of time (typically one night), to a platform that provides continuous health and sleep therapy. The system 400, as previously described, can continuously learn, adjust, adapt, and optimize itself to the individual on a day-by-day basis and provide therapy for OSA never before provided.

### VIII. Monitoring of Physiologic Conditions During Sleep

During sleep, an individual is captive in the sense that the individual is not engaged in another activity, subject to time or deadline demands, or otherwise distracted by surrounding events or demands upon their time. Sleep therefore is an ideal time to monitor the overall health and physiologic state of an individual in a non-intrusive and efficient way. During sleep, a “busy” person has the time to subject themselves to monitoring and collection of information pertaining to their health and well being. During sleep, a person otherwise “uncomfortable” or fearful of visiting a healthcare provider can have collected information pertaining to their health and well-being in the comfort and privacy of their own bedroom.

The systems and methods described herein can be integrated into a home-based health monitoring system 400, of the type shown in FIG. 27. In this arrangement, the person need not have sleep apnea, and the home-based system need not provide apnea therapy. Instead, the home-based health monitoring system serves to sense, monitor, and collect information pertaining to the individual's general health and well being during sleep.

The individual can, for example, be under treatment for a diagnosed medical condition, such as heart arrhythmia, high blood pressure, diabetes, bladder control problems, fecal incontinence, and the like. The system can provide a non-invasive sensor or sensors that sense relevant heath parameters associated with the medical condition and/or general physiologic condition of the individual; for example, operation of an implanted pacemaker, operation of implantable electrical muscle/nerve stimulation device, heart rate, blood pressure, body temperature, and other vital signs, as well as other physiologic parameters described earlier, such as peripheral arterial tone; the level of oxygen in the blood; chest and diaphragm effort, expansion and/or contraction; EEG; EMG (electrical muscle activity); respiration or breathing rate. The system monitors these conditions during sleep and registers and records the sensed conditions through the patient portal 402 linked by land line or internet connection or suitable “tele-med” link to a caregiver portal 404, which processes the information according to pre-programmed rules to provide information and historical data for the individual. The pre-programmed rules can also detect out of bound conditions that help the individual assess their personal health status to facilitate self management and/or alert the individual of the need to consult with a caregiver.

The individual can, on the other hand, be recovering from surgery. In this situation, the monitoring during sleep assesses the individual's recovery.

Alternatively, the individual can be perfectly healthy and still gain benefit from monitoring during sleep. In this situation, the monitoring during sleep assesses the states of the individual's health and well being, without the real time presence of a caregiver. The pre-programmed rules of the caregiver portal 404 can record the sensed conditions, as an assurance to the individual that their health remains good. The pre-programmed rule can detect out of bound conditions and alert the individual to changes in their health status to facilitate their health self management and/or alert the individual of the need to consult with a caregiver.

The pre-programmed rules of the caregiver portal 404 can incorporate clinically created correlations between sleep patterns and physiological conditions sensed during sleep, predictive of the onset of disease states or physiologic dysfunction, such as, for example, coronary artery disease, congestive heart failure, high blood pressure, high blood glucose levels, prostate function, or kidney function. These correlations are developed by the systematic monitoring and study of human physiology during sleep, during which conditions can be detected that could not be detected while the individual is awake. The analysis of physiologic conditions sensed during sleep can serve to provide early detection of disease states or physiologic dysfunction, without the real time presence of a caregiver.

A home-based contact or non-contact health monitoring system just described can, if desired, further incorporate corrective functions that seek to ameliorate out of bound conditions sensed during sleep.

The above-described embodiments of this invention are merely descriptive of its principles and are not to be limited. The scope of this invention instead shall be determined from the scope of the following claims, including their equivalents.

We claim:

1. A system comprising
a generator operative to supply positive air pressure,
a delivery device sized and configured to be worn by an individual in communication with the individual's airway to deliver the positive air pressure from the generator into the airway while sleeping,
a sensing component that senses one or more physical and/or physiologic sleep conditions of the individual to generate a sleep condition output indicative of the physical and/or physiologic sleep condition of the individual,
a monitoring component communicating with the sensing component to compare the sleep condition output with one or more benchmark conditions that correlate to a desired sleep physical and/or physiologic condition, the monitoring component generating an alarm output when a desired physical and/or physiologic sleep condition is absent, and
a corrective action component communicating with the monitoring component including a corrective action element that, in response to the alarm output, affects an operating condition of the positive pressure generator and/or generates at least one sensory or physiologic disturbance and/or alters an orientation or configuration of a sleep surface to influence or alter the physical and/or physiologic sleep condition of the individual to return the individual to a physical and/or physiologic sleep condition that correlates to a desired physical and/or physiologic sleep condition.

2. A system according to claim 1
wherein the sensing component senses a sleeping position.

3. A system according to claim 1
wherein the sensing component comprises one of a gravity-sensitive sensor, a pressure-sensitive sensor, a proximity-sensitive sensor, a magnetic-sensitive sensor, an electronic position-sensitive sensor, a visual position sensor, or an EPT sensor incorporating electronic perception technology (EPT).

4. A system according to claim 1
wherein the sensing component senses the architecture of sounds or vibrations during breathing.

5. A system according to claim 1
wherein the sensing component senses physiologic conditions of the individual.

6. A system according to claim 1
wherein the sensing component is carried by the delivery device.

7. A system according to claim 6
wherein the delivery device comprises a mask.

8. A system according to claim 1
wherein the sensing component is external of the delivery device.

9. A system according to claim 1
wherein, in response to the alarm output, the corrective action element titrates the magnitude of positive air pressure supplied by the generator.

10. A system according to claim 1
wherein, in response to the alarm output, the corrective action element increases the magnitude of positive air pressure supplied by the generator.

11. A system according to claim 1
wherein, in response to the alarm output, the corrective action element ramps the magnitude of positive air pressure supplied by the generator upward according to prescribed increments.

12. A system according to claim 10
wherein, upon return to a physical and/or physiologic sleep condition that correlates to a desired physical and/or physiologic sleep condition, the corrective action element decreases the magnitude of positive air pressure supplied by the generator.

13. A system according to claim 10
wherein, upon return to a physical and/or physiologic sleep condition that correlates to a desired physical and/or physiologic sleep condition, the corrective action element ramps the magnitude of positive air pressure supplied by the generator downward according to prescribed increments.

14. A system according to claim 1
wherein the corrective action element includes a correlation function that compares the magnitude of positive air pressure with termination of the alarm output and iteratively adjusts the maximum positive air pressure according to the correlation.

15. A system according to claim 1
wherein the at least one sensory or physiologic disturbance affects the individual in a tactile, auditory, or other sensory way sufficient to arouse the individual.

16. A system according to claim 1
wherein the at least one sensory or physiologic disturbance affects the individual in a tactile, auditory, or other sensory way that does not awake and/or arouse the individual and/or subconsciously disturb or change or interrupt the sleep state of the individual.

17. A system according to claim 1
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

18. A system according to claim 1
wherein the at least one sensory or physiologic disturbance is introduced into the delivery device.

19. A method comprising
conveying positive air pressure from a positive air pressure generator into the airway of an individual during sleep,
sensing one or more physical and/or physiologic sleep conditions of the individual to generate a sleep condition output indicative of the physical and/or physiologic sleep condition of the individual,
comparing the sleep condition output with one or more benchmark conditions that correlate to a desired sleep physical and/or physiologic condition,
generating an alarm output when a desired physical and/or physiologic sleep condition is absent, and
in response to the alarm output, affecting an operating condition of the positive pressure generator and/or generating at least one sensory or physiologic disturbance and/or altering an orientation or configuration of a sleep surface, to influence or alter the physical and/or physiologic sleep condition of the individual to return the individual to a physical and/or physiologic sleep condition that correlates to a desired physical and/or physiologic sleep condition.

20. A diagnostic home screening system for obstructive breathing conditions comprising
at least one sound sensitive element that senses respiratory sounds made by an individual during a sleep session and that is sized and configured to be placed on or near the individual during the sleep session,
a micro-processing element that is coupled to the sound sensitive element and that is sized and configured to be placed on or near the individual during the sleep session, the micro-processing element including at least one pre-programmed digital sound processing algorithm that processes and registers respiratory sounds sensed by the sound sensitive element over a sleep session; compares the processed respiratory sounds registered during the sleep session to benchmark conditions correlated to a range of obstructive breathing conditions from slight to severe; selects, based upon the comparison, an obstructive breathing condition from the range of obstructive breathing conditions; and generates a diagnostic output indicative of the obstructive breathing condition selected by the pre-programmed digital sound processing algorithm,
a display element that is coupled to the micro-processor and that is sized and configured to visually present the diagnostic output in a format that is directly readable by the individual without interpretation by a doctor, trained sleep professional, and/or analysis out of the home, and
instructions that direct the individual to place the at least one sound sensitive element on or near the individual during the sleep session while at home, to place the micro-processing element on or near the individual during the sleep session while at home, to complete the sleep session while at home with the at least one sound sensitive element and the micro-processing element monitoring the individual's respiratory sounds, and to read the diagnostic output.

21. A system according to claim 20
wherein the diagnostic output is indicative of the site causing an obstructive breathing or snoring condition.

22. A system according to claim 20
wherein the display element comprises one or more lighted indicators.

23. A system according to claim 20
wherein the display element comprises an alpha and/or numeric display.

24. A system according to claim 20
wherein the instructions optionally direct the individual to re-test during a subsequent sleep session while at home.

25. A system according to claim 20
wherein the instructions optionally direct the individual to dispose of the system after use.

26. A method comprising
providing an individual with a diagnostic home screening system for obstructive breathing conditions comprising at least one sound sensitive element that senses respiratory sounds made by the individual during a sleep session and that is sized and configured to be placed on or near the individual during the sleep session, a micro-processing element that is coupled to the sound sensitive element and that is sized and configured to be placed on or near the individual during the sleep session, the micro-processing element including at least one pre-programmed digital sound processing algorithm that processes and registers respiratory sounds sensed by the sound sensitive element over a sleep session; compares the processed respiratory sounds registered during the sleep session to benchmark conditions correlated to a range of obstructive breathing conditions from slight to severe; selects, based upon the comparison, an obstructive breathing condition from the range of obstructive breathing conditions; and generates a diagnostic output indicative of the obstructive breathing condition selected by the pre-programmed digital sound processing algorithm, and a display element that is coupled to the micro-processor and that is sized and configured to visually present the diagnostic output in a format that is directly readable by the individual without interpretation by a doctor or trained sleep professional,
instructing the individual to place the at least one sound sensitive element on or near the individual during the sleep session while at home,
instructing the individual to place the micro-processing element on or near the individual during the sleep session while at home,
instructing the individual to complete the sleep session while at home with the at least one sound sensitive element and the micro-processing element monitoring the individual's respiratory sounds, and
instructing the individual to read the diagnostic output.

27. A method according to claim 26
optionally further instructing the individual to dispose of the system after the sleep session.

28. A method according to claim 26
optionally further instructing the individual to re-test during a subsequent sleep session while at home.

29. A system comprising
an EPT sensing component incorporating electronic perception technology (EPT) sized and configured to provide a sleep status dependent output indicative of the relative sleep state of the individual, and
a monitoring component communicating with the EPT sensing component to receive the sleep status dependent output, the monitoring component including a processing element that compares the sleep status dependent output with one or more benchmark conditions that correlate to a desired sleep state, the processing element generating an alarm output when the individual is not in a desired sleep state.

30. A system according to claim 29
further including a corrective action component communicating with the monitoring component including a corrective action element that, in response to the alarm output, generates an output to influence or alter the individual's sleep state to return the individual to a sleep state that correlates to a desired sleep state.

31. A system according to claim 29
wherein the output comprises at least one sensory or physiologic disturbance.

32. A system according to claim 31
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

33. A system according to claim 29
wherein the sleep state includes sleep position.

34. A system according to claim 29
wherein the sleep state includes existence of restless leg syndrome; sleep walking; going to and from the bathroom to urinate; tossing, turning, head movement, eye movements due to sleeplessness; GERD-based movements; periodic limb movements; or overall sleep habits.

35. A system according to claim 29
wherein the EPT sensing component comprises at least one of CMOS-based Time-of-flight [ToF], a stereo camera, and structured light.

36. A method comprising
providing an EPT sensing component incorporating electronic perception technology (EPT) sized and configured to provide a sleep status dependent output indicative of the relative sleep state of the individual,
instructing the individual to operate the EPT sensing component to observe the sleep state of the individual during a sleep session,
comparing the sleep status dependent output with one or more benchmark conditions that correlate to a desired sleep state, and
generating an alarm output when the individual is not in a desired sleep state.

37. A method according to claim 36
further including generating an output in response to the alarm output to influence or alter the individual's sleep state to return the individual to a sleep state that correlates to a desired sleep state.

38. A method according to claim 37
wherein the output comprises at least one sensory or physiologic disturbance.

39. A method according to claim 38
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

40. A method according to claim 36
wherein the sleep state includes sleep position.

41. A method according to claim 36
wherein the sleep state includes existence of restless leg syndrome; sleep walking; going to and from the bathroom to urinate; tossing, turning, head movement, eye movements due to sleeplessness; GERD-based movements; periodic limb movements; or overall sleep habits.

42. A method according to claim 36
wherein the EPT sensing component comprises at least one of CMOS-based Time-of-flight [ToF], a stereo camera, and structured light.

43. A system comprising
a first sensing component that senses a first physical and/or physiologic sleep condition of an individual to generate a first sleep condition output indicative of the first physical and/or physiologic sleep condition of the individual,
a second sensing component that senses a second physical and/or physiologic sleep condition of an individual to generate a second sleep condition output indicative of the second physical and/or physiologic sleep condition of the individual, and
a monitoring component communicating with the first and second sensing components to compare the first sleep condition output with one or more benchmark conditions that correlate to a first desired sleep physical and/or physiologic condition; the monitoring component also comparing the second sleep condition output with one or more benchmark conditions that correlate to a second desired sleep physical and/or physiologic condition, the monitoring component generating an alarm output only when both the first and second desired physical and/or physiologic sleep conditions are absent.

44. A system according to claim 43
further including a corrective action component communicating with the monitoring component including a corrective action element that, in response to the alarm output, generates an output to influence or alter at least one the first and second physical and/or physiologic sleep conditions of the individual to return the individual to a physical and/or physiologic sleep condition that correlates to a desired physical and/or physiologic sleep condition.

45. A system according to claim 43
wherein one of the first and second sensing components senses a sleeping position.

46. A system according to claim 43
wherein the sensing component that senses a sleeping position comprises one of a gravity-sensitive sensor, a pressure-sensitive sensor, a proximity-sensitive sensor, a magnetic-sensitive sensor, an electronic position-sensitive sensor, a visual position sensor, and an EPT sensing component.

47. A system according to claim 43
wherein one of the first and second sensing components senses the architecture of sounds or vibrations during breathing.

48. A system according to claim 43
wherein one of the first and second sensing components senses physiologic conditions of the individual.

49. A method comprising
sensing a first physical and/or physiologic sleep condition of an individual to generate a first sleep condition output indicative of the first physical and/or physiologic sleep condition of the individual,
sensing a second physical and/or physiologic sleep condition of an individual to generate a second sleep condition output indicative of the second physical and/or physiologic sleep condition of the individual,
comparing the first sleep condition output with one or more benchmark conditions that correlate to a first desired sleep physical and/or physiologic condition,
comparing the second sleep condition output with one or more benchmark conditions that correlate to a second desired sleep physical and/or physiologic condition, and
generating an alarm output only when both the first and second desired physical and/or physiologic sleep conditions are absent.

50. A method according to claim 49
further including generating, in response to the alarm output, an output to influence or alter at least one the first and second physical and/or physiologic sleep conditions of the individual to return the individual to a physical and/or physiologic sleep condition that correlates to a desired physical and/or physiologic sleep condition.

51. A method according to claim 49
wherein one of the first and second physical and/or physiologic sleep conditions of the individual includes a sleeping position.

52. A method according to claim 49
wherein one of the first and second physical and/or physiologic sleep conditions of the individual includes the architecture of sounds or vibrations during breathing.

53. A method according to claim 49
wherein one of the first and second physical and/or physiologic sleep conditions of the individual includes a physiologic condition of the individual.

54. A system comprising
a sensing component that senses at least one physical and/or physiologic sleep condition of an individual to generate a sleep condition output indicative of the physical and/or physiologic sleep condition of the individual,
a monitoring component communicating with the first sensing component to compare the sleep condition output with one or more benchmark conditions that correlate to a desired sleep physical and/or physiologic condition and to generate an alarm output when the desired physical and/or physiologic sleep condition is absent,
a corrective action component communicating with the monitoring component including a corrective action element that, in response to the alarm output, selects a corrective action output to influence or alter the physical and/or physiologic sleep condition of the individual to return the individual to a physical and/or physiologic sleep condition that correlates to a desired physical and/or physiologic sleep condition, and
a learning function component that iteratively adjusts the selection of the corrective action output according to the sensed physical and/or physiologic sleep condition of the individual to optimize the return of the individual to a physical and/or physiologic sleep condition that correlates to a desired physical and/or physiologic sleep condition.

55. A system according to claim 54
wherein the at least one physical and/or physiologic sleep condition of the individual includes a sleeping position.

56. A system according to claim 54
wherein the at least one physical and/or physiologic sleep condition of the individual includes the architecture of sounds or vibrations during breathing.

57. A system according to claim 54
wherein the at least one physical and/or physiologic sleep condition of the individual includes a physiologic condition of the individual.

58. A system according to claim 57
wherein the at least one physical and/or physiologic sleep condition of the individual includes a predictor, presence, absence, or onset of an apnea sleep event.

59. A system according to claim 54
wherein the selected corrective action output includes at least one sensory or physiologic disturbance.

60. A system according to claim 59
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

61. A system according to claim 59
wherein the learning function component titrates the magnitude of the at least one sensory or physiologic disturbance to minimize arousal and/or disruption of the sleep state.

62. A system according to claim 54
further including a variable sleep surface,
wherein the selected corrective action output varies the variable sleep surface.

63. A system according to claim 62
wherein the learning function component adjust the variance of the variable sleep surface.

64. A system according to claim 54
wherein the learning function iteratively adjusts the selection of the type and/or magnitude/amplitude and/or duration of corrective action output to minimize arousal and/or disruption of the sleep state while optimizing the sleep condition.

65. A system according to claim 54
wherein the learning function iteratively adjusts the selection of the corrective action output according to the individual's response to the corrective action output to control and/or improve their physical and/or physiologic sleep condition and maintain a desired physical and/or physiologic sleep condition.

66. A method comprising
sensing at least one physical and/or physiologic sleep condition of an individual to generate a sleep condition output indicative of the physical and/or physiologic sleep condition of the individual,
comparing the sleep condition output with one or more benchmark conditions that correlate to a desired sleep physical and/or physiologic condition and to generate an alarm output when the desired physical and/or physiologic sleep condition is absent,
selecting, in response to the alarm output, a corrective action output to influence or alter the physical and/or physiologic sleep condition of the individual to return the individual to a physical and/or physiologic sleep condition that correlates to a desired physical and/or physiologic sleep condition, and
iteratively adjusting the selection of the corrective action output according to the sensed physical and/or physiologic sleep condition of the individual to optimize the return of the individual to a physical and/or physiologic sleep condition that correlates to a desired physical and/or physiologic sleep condition.

67. A method according to claim 66
wherein the at least one physical and/or physiologic sleep condition of the individual includes a sleeping position.

68. A method according to claim 66
wherein the at least one physical and/or physiologic sleep condition of the individual includes the architecture of sounds or vibrations during breathing.

69. A method according to claim 66
wherein the at least one physical and/or physiologic sleep condition of the individual includes a physiologic condition of the individual.

70. A method according to claim 66
wherein the at least one physical and/or physiologic sleep condition of the individual includes a predictor, presence, absence, or onset of an apnea sleep event.

71. A method according to claim 66
wherein the selected corrective action output includes at least one sensory or physiologic disturbance.

72. A method according to claim 71
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

73. A method according to claim 71
wherein iteratively adjusting the selection of the corrective action output includes titrating the magnitude of the at least one sensory or physiologic disturbance to minimize arousal and/or disruption of the sleep state.

74. A method according to claim 66
wherein the selected corrective action output adjusts a variable sleep surface.

75. A method according to claim 74
wherein iteratively adjusting the selection of the corrective action output includes varying the adjustment of the variable sleep surface.

76. A method according to claim 66
wherein the learning function iteratively adjusts the selection of the corrective action output to minimize arousal and/or disruption of the sleep state.

77. A method according to claim 66
wherein the learning function iteratively adjusts the selection of the corrective action output according to the individual's response to the corrective action output to control and/or improve their physical and/or physiologic sleep condition and maintain a desired physical and/or physiologic sleep condition.

78. A system comprising
a sound sensing component comprising a sound sensor sized and configured to be implanted in an individual to provide a sound dependent output indicative of an architecture of breathing sounds and/or vibrations made by the individual while asleep.

79. A system according to claim 78
further including a monitoring component communicating with the sound sensing component to receive the sound dependent output, the monitoring component including a processing element that compares the sound dependent output with one or more benchmark conditions that correlate to a desired architecture of sound and/or vibrations, the processing element generating an alarm output when the sound dependent output is not a desired architecture of sound and/or vibrations.

80. A system according to claim 79
further including a corrective action component communicating with the monitoring component including a corrective action element that, in response to the alarm output, generates a corrective action output to influence or alter the individual's architecture of breathing sounds and/or vibrations made by the individual to return the individual's architecture to an architecture that correlates to a desired architecture.

81. A system according to claim 80
wherein the corrective action output includes at least one sensory or physiologic disturbance.

82. A system according to claim 81
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

83. A method comprising
providing a sound sensing component comprising a sound sensor sized and configured to be implanted in an individual to provide a sound dependent output indicative of an architecture of breathing sounds and/or vibrations made by the individual while asleep, and
implanting the sound sensing component.

84. A method according to claim 83
further including monitoring the sound dependent output including comparing the sound dependent output with one or more benchmark conditions that correlate to a desired architecture of sound and/or vibrations and generating an alarm output when the sound dependent output is not a desired architecture of sound and/or vibrations.

85. A method according to claim 84
further including, in response to the alarm output, generating a corrective action output to influence or alter the individual's architecture of breathing sounds and/or vibrations made by the individual to return the individual's architecture to an architecture that correlates to a desired architecture.

86. A method according to claim 85
wherein the corrective action output includes at least one sensory or physiologic disturbance.

87. A method according to claim 86
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

88. A system comprising
a position sensing component comprising a position sensor sized and configured to be implanted in an individual to provide a position dependent output indicative of the relative sleep position of the individual.

89. A system according to claim 88
further including a monitoring component communicating with the position sensor to receive the position dependent output, the monitoring component including a processing element that compares the position dependent output with one or more benchmark conditions that correlate to a desired sleep position, the processing element generating an alarm output when the individual is not in a desired sleep position.

90. A system according to claim 89
further including a corrective action component communicating with the monitoring component including a corrective action element that, in response to the alarm output, generates a corrective action output to influence or alter the individual's sleep position to return the individual to a sleep position that correlates to a desired sleep position.

91. A system according to claim 90
wherein the corrective action output includes at least one sensory or physiologic disturbance.

92. A system according to claim 91
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

93. A method comprising
providing a position sensing component comprising a position sensor sized and configured to be implanted in an individual to provide a position dependent output indicative of the relative sleep position of the individual, and
implanting the position sensing component.

94. A method according to claim 93
further monitoring the position dependent output including comparing the position dependent output with one or more benchmark conditions that correlate to a desired sleep position, the processing element generating an alarm output when the individual is not in a desired sleep position.

95. A method according to claim 94
further including, in response to the alarm output, generating a corrective action output to influence or alter the individual's sleep position to return the individual to a sleep position that correlates to a desired sleep position.

96. A method according to claim 95
wherein the corrective action output includes at least one sensory or physiologic disturbance.

97. A method according to claim 96
wherein the at least one sensory or physiologic disturbance comprises at least one of smell, sound, vibration, and taste, light, vibration, temperature, nerve or electrical stimulation of muscles, energy stimulation (ultrasound, radio-frequency, etc), airflow, puffs of air, and moisture.

98. A system comprising
an implant body sized and configured to be implanted in an individual,
a sensing component carried by the implant body to provide an sensed output indicative of a sleep condition of an individual while asleep, and
a monitoring component communicating with the sensing component and carried by the implant body to receive the sensed output, the monitoring component including a processing element that compares the sensed output with one or more benchmark conditions that correlate to a desired sleep condition, the processing element generating an alarm output when the individual is not in a desired sleep condition.

99. A system according to claim 98
further including a corrective action component communicating with the monitoring component and carried by the implant body including a corrective action element that, in response to the alarm output, generates a corrective action output to influence or alter the individual's sleep position to return the individual to a sleep position that correlates to a desired sleep position.

100. A system comprising
a sensing component sized and configured to provide an sensed output indicative of a sleep condition of an individual while asleep,
a corrective action component communicating with the sensing component being sized and configured to be implanted in the individual, the corrective action component including a corrective action element that, in response to the sensed output, generates a corrective action output to influence or alter the individual's sleep position to return the individual to a sleep position that correlates to a desired sleep position.

101. A system comprising
a sensing component sized and configured to provide an sensed output indicative of a sleep condition of an individual while asleep, and
a monitoring component communicating with the sensing component being sized and configured to be implanted in the individual, the monitoring component including a processing element that compares the sensed output with one or more benchmark conditions that correlate to a desired sleep condition, the processing element generating an alarm output when the individual is not in a desired sleep condition.
